(19) United States
(12) Patent Application Publication
(10) Pub. No.: US 2022/0306757 A1
(43) Pub. Date: Sep. 29, 2022

(54) BCMA ANTIBODY

(71) Applicants: HANGZHOU SUMGEN BIOTECH CO., LTD., Hangzhou (CN); SUMGEN MAB (BEIJING) BIOTECH CO., LTD., Beijing (CN)

(72) Inventors: Ming LV, Hangzhou (CN); Xiaoran DING, Hangzhou (CN); Shiwei MIAO, Hangzhou (CN); Bin TAN, Hangzhou (CN); Xuegong WANG, Hangzhou (CN)

(73) Assignees: HANGZHOU SUMGEN BIOTECH CO., LTD., Hangzhou (CN); SUMGEN MAB (BEIJING) BIOTECH CO., LTD., Beijing (CN)

(21) Appl. No.: 17/636,782

(22) PCT Filed: Aug. 19, 2020
(86) PCT No.: PCT/CN2020/110068

§ 371 Date: Feb. 18, 2022

(30) Foreign Application Priority Data
Aug. 20, 2019 (CN) 201910767933.6

(51) Int. Cl. C07K 16/28; A61K 47/68; A61P 35/00; G01N 33/574

(52) U.S. Cl. CPC C07K 16/2878; A61K 47/6849; A61P 35/00; G01N 33/57492; A61K 2039/505

(57) ABSTRACT

Provided is an antibody or an antigen-binding fragment thereof having one or more properties selected from the group consisting of: 1) capable of binding to a BCMA protein with a K_{D} value of 1.8×10⁻⁹ M or lower; 2) capable of specifically recognizing a BCMA protein expressed on cell surfaces; 3) capable of being internalized by cells; and 4) capable of inhibiting tumors and/or tumor cell proliferation. The antibody can also be used to construct immunoconjugates. The antibody and immunoconjugates can all prevent or treat tumors.

### FIELD OF THE INVENTION

The present application relates to an antibody binding to a BCMA protein and application thereof. The antibody can specifically recognize and bind to a BCMA protein, and mediate internalization after binding to the BCMA protein expressed on the cell surface. The antibody can inhibit the growth and/or proliferation of tumors or tumor cells.

### BACKGROUND OF THE INVENTION

B cell maturation antigen BCMA (CD269 or TNFRSF17) is a member of the tumor necrosis factor superfamily, which is mainly expressed on plasma cells and some mature B cells. Human BCMA is a type III transmembrane protein composed of 184 amino acid residues. BCMA can specifically bind to a proliferation-inducing ligand (APRIL) and B cell activating factor (BAFF) to activate NF-κB and MAPK8/JNK signaling pathways; it can also bind to a variety of TRAF family members to promote the survival of B cells in different developmental stages, and participate in the regulation of humoral immunity, B cell development and homeostasis.

It is reported that, BCMA is highly expressed on the surface of a variety of tumor cells, such as multiple myeloma, Hodgkin lymphoma, acute lymphoblastic leukemia (ALL), but not expressed in hematopoietic stem cells and other normal tissue cells, so it is an ideal target for the targeted treatment of cancers.

In view of the therapeutic potential of BCMA, it is necessary to prepare an antibody that specifically binds to the BCMA protein.

### SUMMARY OF THE INVENTION

The present application provides an antibody binding to a BCMA protein and application thereof. The antibody or the antigen-binding fragment thereof of the present application has one or more of the following properties: 1) specifically binding to a BCMA protein at a relatively high affinity; 2) specifically recognizing a BCMA protein expressed on cell surfaces; 3) capable of being internalized by cells, especially being internalized by tumor cells; 4) inhibiting the growth and/or proliferation of tumors or tumor cells; 5) can be used to construct an immunoconjugate, and the immunoconjugate can also inhibit the growth and/or proliferation of tumors or tumor cells. The present application also provides a preparation method and an application of the antibody.

In one aspect, the present application provides an antibody or an antigen-binding fragment thereof, which includes at least one CDR in an antibody light chain variable region VL, the VL includes an amino acid sequence as set forth in SEQ ID NO: 7.

In some embodiments, the antibody or the antigen-binding fragment thereof includes at least one CDR in an antibody heavy chain variable region VH, wherein the VH includes an amino acid sequence as set forth in SEQ ID NO: 8.

In some embodiments, the antibody or the antigen-binding fragment thereof has one or more properties selected from the group consisting of: 1) capable of binding to a BCMA protein at a K_{D} value of 1.8×10⁻⁹ M or below; 2) capable of specifically recognizing a BCMA protein expressed on cell surfaces; 3) capable of being internalized by cells; 4) inhibiting the proliferation of tumors and/or tumor cells.

In some embodiments, the BCMA protein is a human BCMA protein.

In some embodiments, the antibody is selected from the group consisting of a monoclonal antibody, a single-chain antibody, a chimeric antibody, a multispecific antibody, a humanized antibody and a fully human antibody.

In some embodiments, the antigen-binding fragment is selected from the group consisting of Fab, Fab′, F(ab)₂, F(ab′)₂, Fv and ScFv fragments.

In some embodiments, the antibody or the antigen-binding fragment thereof competes with the reference antibody for binding to the BCMA protein, wherein the reference antibody includes a light chain variable region and a heavy chain variable region, the light chain variable region of the reference antibody includes LCDR1, LCDR2 and LCDR3, the LCDR1 includes an amino acid sequence as set forth in SEQ ID NO:1; the LCDR2 includes an amino acid sequence as set forth in SEQ ID NO: 2; the LCDR3 includes an amino acid sequence as set forth in SEQ ID NO: 3; the heavy chain variable region of the reference antibody includes HCDR1, HCDR2 and HCDR3, the HCDR1 includes an amino acid sequence as set forth in SEQ ID NO: 4; the HCDR2 includes an amino acid sequence as set forth in SEQ ID NO: 5; the HCDR3 includes an amino acid sequence as set forth in SEQ ID NO: 6.

In some embodiments, the light chain variable region of the reference antibody includes an amino acid sequence as set forth in SEQ ID NO: 7, and the heavy chain variable region of the reference antibody includes an amino acid sequence as set forth in SEQ ID NO: 8. In some embodiments, the light chain of the reference antibody includes an amino acid sequence as set forth in SEQ ID NO: 9; and the heavy chain of the reference antibody includes an amino acid sequence as set forth in SEQ ID NO: 10.

In some embodiments, the reference antibody includes an antibody SG1116.

In some embodiments, the antibody includes an antibody light chain or a fragment thereof. In some embodiments, the antibody light chain or the fragment thereof includes LCDR1, and the LCDR1 includes an amino acid sequence as set forth in SEQ ID NO: 1. In some embodiments, the antibody light chain or the fragment thereof includes LCDR2, and the LCDR2 includes an amino acid sequence as set forth in SEQ ID NO: 2. In some embodiments, the antibody light chain or the fragment thereof includes LCDR3, and the LCDR3 includes an amino acid sequence as set forth in SEQ ID NO: 3.

In some embodiments, the antibody light chain or the fragment thereof includes a light chain variable region VL, and the light chain variable region VL includes an amino acid sequence as set forth in SEQ ID NO: 7.

In some embodiments, the antibody light chain or the fragment thereof also includes a human constant region. In some embodiments, the human constant region includes a human Igκ constant region.

In some embodiments, the antibody light chain or the fragment thereof includes an amino acid sequence as set forth in SEQ ID NO: 9.

In some embodiments, the antibody includes an antibody heavy chain or a fragment thereof. In some embodiments, the antibody heavy chain or the fragment thereof includes HCDR1, and the HCDR1 includes an amino acid sequence as set forth in SEQ ID NO: 4. In some embodiments, the antibody heavy chain or the fragment thereof includes HCDR2, and the HCDR2 includes an amino acid sequence as set forth in SEQ ID NO: 5. In some embodiments, the antibody heavy chain or the fragment thereof includes HCDR3, and the HCDR3 includes an amino acid sequence as set forth in SEQ ID NO: 6.

In some embodiments, the antibody heavy chain or the fragment thereof includes a heavy chain variable region VH, and the heavy chain variable region VH includes an amino acid sequence as set forth in SEQ ID NO: 8.

In some embodiments, the antibody heavy chain or the fragment thereof also includes a human constant region. In some embodiments, the human constant region includes a human IgG constant region. In some embodiments, the IgG constant region includes a human IgG1 constant region.

In some embodiments, the antibody heavy chain or the fragment thereof includes an amino acid sequence as set forth in SEQ ID NO: 10.

In some embodiments, the antibody includes an antibody SG1116.

In another aspect, the present application provides one or more isolated nucleic acid molecules, which encode the antibody or the antigen-binding fragment thereof.

In another aspect, the present application provides one or more vectors, which include the nucleic acid molecules.

In another aspect, the present application provides cells, which include the nucleic acid molecules or the vectors.

In another aspect, the present application provides a method of preparing the antibody or the antigen-binding fragment thereof, which includes culturing the cell under conditions allowing the expression of the antibody or the antigen-binding fragment thereof.

In another aspect, the present application provides an immunoconjugate, which includes the antibody or the antigen-binding fragment thereof.

In some embodiments, the immunoconjugate also includes at least one additional reagent selected from the group consisting of a chemotherapeutic agent, a radioactive element, a cell growth inhibitor and a cytotoxic agent. In some embodiments, in the immunoconjugate, the antibody or the antigen-binding fragment thereof is directly or indirectly linked to the at least one additional reagent. In some embodiments, the indirect linking in the immunoconjugate includes linking through a linking molecule, and the linking molecule includes a SMCC linker.

In some embodiments, the at least one additional reagent includes maytansine or a derivative thereof. In some embodiments, the maytansine derivative includes a maytansine derivative DM1.

In another aspect, the present application provides a pharmaceutical composition, which includes the antibody or the antigen-binding fragment thereof, the immunoconjugate, or the cell, as well as optionally a pharmaceutically acceptable adjuvant.

In another aspect, the present application provides a use of the antibody or the antigen-binding fragment thereof, or the immunoconjugate in the preparation of a medicament, the medicament is used for preventing or treating a tumor.

In some embodiments, the tumor includes a BCMA positive tumor. In some embodiments, the tumor includes a non-solid tumor. In some embodiments, the tumor includes a tumor selected from the group consisting of myeloma, lymphoma and hematologic malignancy.

In another aspect, the present application provides the antibody or the antigen-binding fragment thereof, or the immunoconjugate, which is used for preventing or treating a tumor.

In some embodiments, the tumor includes a BCMA positive tumor. In some embodiments, the tumor includes a non-solid tumor. In some embodiments, the tumor includes a tumor selected from the group consisting of myeloma, lymphoma and hematologic malignancy.

In another aspect, the present application provides a method of preventing or treating a tumor in a subject in need thereof, which includes administering to the subject the antibody or the antigen-binding fragment thereof, the immunoconjugate, or the pharmaceutical composition.

In some embodiments, the tumor includes a BCMA positive tumor. In some embodiments, the tumor includes a non-solid tumor. In some embodiments, the tumor includes a tumor selected from the group consisting of myeloma, lymphoma and hematologic malignancy.

In another aspect, the present application provides the antibody or the antigen-binding fragment thereof, which is used for diagnosing diseases or conditions related to the expression of the BCMA protein.

In another aspect, the present application provides a use of the antibody or the antigen-binding fragment thereof in the preparation of a diagnostic agent, wherein the diagnostic agent is used for diagnosing diseases or conditions related to the expression of the BCMA protein.

In another aspect, the present application provides a method of determining diseases or conditions related to the expression of the BCMA protein in a subject, which includes: contacting a sample derived from the subject with the antibody or the antigen-binding fragment thereof, and determining the presence and/or content of substances in the sample which are capable of specifically binding to the antibody or the antigen-binding fragment thereof.

Those skilled in the art can easily perceive other aspects and advantages of the present disclosure from the detailed description below. In the following detailed description, only exemplary embodiments of the present disclosure are shown and described. As those skilled in the art will recognize, the content of the present disclosure enables those skilled in the art to make changes to the disclosed specific embodiments without departing from the spirit and scope of the invention involved in the present application. Correspondingly, the drawings and descriptions in the specification of the present application are merely exemplary, rather than restrictive.

### BRIEF DESCRIPTION OF THE DRAWINGS

The specific features of the invention involved in the present application are shown in the appended claims. The characteristics and advantages of the invention involved in the present application can be better understood by referring to the exemplary embodiments and the accompanying drawings described in detail below. A brief description of the drawings is as follows:

FIG. 1 shows the binding activity of the antibody of the present application to BCMA antigen;

FIG. 2 shows the ability of the antibody of the present application to specifically recognize BCMA antigen;

FIG. 3 shows the expression of BCMA antigen on the cell surface;

FIGS. 4A-4B show the results of the recognition of the antigen BCMA on the tumor cell surface by the antibody of the present application;

FIGS. 5A-5B show the results of the antibody of the present application on the internalization activity of tumor cells;

FIGS. 6A-6B show the results of the antibody of the present application in inhibiting the tumor activity in vivo;

FIG. 7 shows the results of the immunoconjugate of the present application in inhibiting the proliferation of tumor cells in vivo;

FIGS. 8A-8B show the results of the immunoconjugate of the present application in inhibiting the tumor activity in vivo.

### DETAILED DESCRIPTION OF THE EMBODIMENTS

The implementation of the present application will be illustrated in the following specific examples, and other advantages and effects of the present application will be easily known by those familiar with this technology from the content disclosed in the specification.

In the present application, the term “antibody” generally refers to an immunoglobulin or a fragment or derivative thereof, including any polypeptides that include an antigen binding site, no matter whether it is produced in vitro o in vivo. The term includes, but is not limited to, polyclonal, monoclonal, mono-specific, multi-specific, non-specific, humanized, single-chain, chimeric, synthetic, recombinant, hybrid, mutated and grafted antibodies. Unless otherwise modified by a term “complete”, as in “complete antibody”, for the purposes of the present invention, the term “antibody” also includes antibody fragments, such as Fab, F(ab′)₂, Fv, scFv, Fd, dAb and other antibody fragments that retain the antigen binding functions (i.e., specifically binding to, e.g., OX40 or PD-L1). In general, such fragments should include antigen-binding domains. The basic 4-chain antibody unit is a heterotetrameric glycoprotein composed of two identical light (L) chains and two identical heavy (H) chains. The IgM antibody is composed of 5 basic heterotetrameric units and another polypeptide called J chain, and contains 10 antigen-binding sites; while the IgA antibody includes 2-5 basic 4-chain units that can be polymerized with the J chain to form a multivalent combination. In terms of IgG, the 4-chain unit is generally about 150,000 Daltons. Each L chain is linked to the H chain through a covalent disulfide bond, while two H chains are linked to each other through one or more disulfide bonds depending on the isotype of the H chain. Each H and L chain also has regularly spaced intra-chain disulfide bridges. Each H chain has a variable domain (VH) at the N-terminus, which is followed by three constant domains (CH) for each of α and γ chains, and followed by four CH domains for μ and ε isotypes. Each L chain has a variable domain (VL) at the N-terminus, and has a constant domain at the other terminus. VL corresponds to VH, and CL corresponds to the first constant domain (CH1) of the heavy chain. Specific amino acid residues are considered to form an interface between the light chain and heavy chain variable domains. VH is paired with VL to form a single antigen-binding site. For the structures and properties of different kinds of antibodies, see for example Basic and Clinical Immunology, 8th Edition, Daniel P. Sties, Abba 1. Terr and Tristram G. Parsolw (eds), Appleton & Lange, Norwalk, Conn., 1994, Page 71 and Chapter 6. L chains from any vertebrate species can be classified into one of two distinct types based on the amino acid sequences of their constant domains, called kappa and lambda. Depending on the amino acid sequence of its heavy chain (CH) constant domain, immunoglobulin can be classified into different types or isotypes. There are five types of immunoglobulin: IgA, IgD, IgE, IgG and IgM, which have heavy chains named α, δ, ε, γ and μ, respectively. Based on the relatively small differences in terms of CH sequence and function, the γ and α types are further divided into sub-types. For example, human expresses the following subtypes: IgG1, IgG2A, IgG2B, IgG3, IgG4, IgA1 and IgK1.

In the present application, the term “CDR” generally refers to an area of an antibody variable domain, of which the sequence is highly variable and/or forms a structure-defining ring. In general, an antibody includes six CDRs; three in VH (HCDR1, HCDR2, HCDR3), and three in VL (LCDR1, LCDR2, LCDR3). In native antibodies, HCDR3 and LCDR3 exhibit the most diversity of the six CDRs, and in particular, HCDR3 is considered to play a special role in conferring a fine specificity to the antibody. See, for example, Xu et al, Immunity 13:37-45 (2000); Johnson and Wu, in Methods in Molecular Biology 248:1-25 (Lo, ed., Human Press, Totowa, N.J., 2003). In fact, naturally occurring camel antibodies only composed of heavy chains function normally and are stable in the absence of light chains. See, for example, Hamers-Casterman et al., Nature 363:446-448 (1993); Sheriff et al, Nature Struct. Biol. 3:733-736 (1996). For example, in the present application, the OX40 binding portion may include a heavy chain variable domain VH1, and the VH1 may include three CDRs (e.g., H1CDR1, H1CDR2 and H1CDR3), and it may further include a light chain variable domain VL1, and the VL1 may include three CDRs (e.g., L1CDR1, L1CDR2 and L1CDR3). For another example, in the present application, the PD-L1 binding portion may include a heavy chain variable domain VH2, and the VH2 may include three CDRs (e.g., H2CDR1, H2CDR2 and H2CDR3).

In the art, the CDRs of an antibody can be classified by a variety of methods, for example, 1) Kabat definition rules based on sequence variability (Wu and Kabat, J Exp Med 132:211-50, 1970; Kabat et al., Sequences of Proteins of Immunological Interest, Ed. 5, Public Health Service, National Institutes of Health, Bethesda, Md., 1991), 2) Chothia definition rules based on the location of structural loop regions (Al-Lazikani et al., J Mol Biol 273:927-48, 1997), 3) AbM definition rules using AbM antibody modeling software of Oxford Molecular to weigh the above two rules, 4) Contact definition rules based on the analysis of the obtained complex crystal structure. These methods of labeling CDRs can be summarized in Table 1 below.

TABLE 1
Classification method of CDR

| | CCG | Kabat | AbM | Chothia | Contact |
|---|---|---|---|---|---|
| | Definition | Definition | Definition | Definition | Definition |
| Light chain | L24-L34 | L24-L34 | L24-L34 | L24-L34 | L30-L36 |
| CDR1 | | | | | |
| Light chain | L50-L56 | L50-L56 | L50-L56 | L50-L56 | L45-L55 |
| CDR2 | | | | | |
| Light chain | L89-L97 | L89-L97 | L89-L97 | L89-L97 | L89-L96 |
| CDR3 | | | | | |
| Heavy chain | H26-H35 | H31-H35 | H26-H35 | H26-H32 | H30-H35 |
| CDR1 | | | | | |
| Heavy chain | H50-H65 | H50-H65 | H50-H58 | H52-H56 | H47-H58 |
| CDR2 | | | | | |
| Heavy chain | H95-H102 | H95-H102 | H95-H102 | H95-H102 | H93-H101 |
| CDR3 | | | | | |

Where, Laa-Lbb may refer to the amino acid sequence from position aa to position bb starting from the N-terminus of the antibody light chain; Haa-Hbb may refer to the amino acid sequence from position aa to position bb starting from the N-terminus of the antibody heavy chain. For example, L24-L34 may refer to the amino acid sequence from position 24 to position 34 starting from the N-terminus of the antibody light chain; H26-H35 may refer to the amino acid sequence from position 26 to position 35 starting from the N-terminus of the antibody heavy chain. In the present application, the Kabat definition rules are used to determine the amino acid residues in the variable domain sequence and the full-length antibody sequence.

In the present application, the term “antigen-binding fragment” generally refers to one or more fragments of an antibody that function to specifically bind to an antigen. The antigen-binding function of an antibody can be achieved by a full-length fragment of the antibody. The antigen-binding function of an antibody can also be achieved through the following: heavy chain including fragments of Fv, ScFv, dsFv, Fab, Fab′ or F (ab′)₂, or light chain including fragments of Fv, ScFv, dsFv, Fab, Fab′ or F (ab′)₂. (1) Fab fragment, that is, a univalent fragment composed of VL, VH, CL and CH domains; (2) F(ab′)₂ fragment, a divalent fragment including two Fab fragments linked through a disulfide bond at the hinge region; (3) a Fd fragment composed of VH and CH domains; (4) a Fv fragment composed of the VL and VH domains of one arm of an antibody; (5) a dAb fragment composed of VH domains (Ward et al, (1989) Nature 341: 544-546); (6) an isolated complementary determining region (CDR) and (7) a combination of two or more isolated CDRs which may be optionally linked through linkers. In addition, it may also include a univalent single-chain moleculeFv (scFv) formed by the pairing of VL and VH (see Bird et al (1988) Science 242: 423-426; as well as Huston et al (1988) Proc. Natl. Acad. Sci. 85: 5879-5883). The “antigen binding portion” can also include a fusion protein including an immunoglobulin. For example, the fusion protein can include a binding domain selected from the group consisting of: (1) a binding domain polypeptide fused with the immunoglobulin hinge region polypeptide; (2) an immunoglobulin heavy chain CH2 constant region fused with the hinge region; and/or (3) an immunoglobulin heavy chain CH3 constant region fused with the CH2 constant region.

In the present application, the term “BCMA” can be interchangeably used with “CD269”, “BCM” and “TNFRSF17” and generally refers to B cell maturation antigen. For example, human BCMA is a protein with a length of 184 amino acids encoded by a 994-nucleotide primary mRNA transcript (NM_001192.2). The amino acid sequence of human BCMA is represented by UniPretKB accession number Q02223. In the present application, the term “BCMA” may include proteins containing mutations, for example, it may include proteins containing point mutations, fragments, insertions, deletions, and splice variants of full-length wild-type BCMA. In the present application, the term “BCMA” may also include a portion of a complete BCMA protein, as long as the relevant biological activity is retained.

In the present application, the term “K_{D}” can be interchangeably used with “KD”, and generally refers to the dissociation equilibrium constant of a specific antibody-antigen interaction at a unit of M (mol/L). KD can be calculated from the concentrations of the substance AB, as well as substance A and substance B dissociated therefrom: KD=c (A)*c (B)/c (AB). It can be known from the equation that the greater the KD value, the more the dissociation, indicating that the weaker the affinity between substances A and B; otherwise, the smaller the KD value, the less the dissociation, indicating that the stronger the affinity between substances A and B.

In the present application, the term “internalization” generally refers to an exogenous substance (e.g., protein, nucleic acid) binding to the cell and entering the cell without the help of other external reagents or conditions (e.g., transfection, electroporation, microinjection, protoplast fusion, etc.). In some cases, internalization may be a process in which an antibody or an antigen-binding fragment thereof or polypeptide specifically binds to a receptor on the cell surface to form a receptor-antibody complex, and then enters the cell through endocytosis mediated by the receptor. In this case, such an antibody or an antigen-binding fragment thereof (e.g. Fab fragment) can become an internalized antibody. The internalized antibody may serve as a carrier for targeted delivery of drugs, enzymes or DNA. In some cases, the internalization can inhibit the proliferation of tumor cells. For example, the internalized antibody can be used to couple anti-tumor chemotherapeutics, radioactive elements, cell growth inhibitors and cytotoxic agents, and as candidate molecules for tumor biotherapy.

In the present application, the term “tumor” generally refers to a physiological condition typically characterized by dysregulation of cell proliferation or survival. The tumor may include all known cancers and tumor conditions, regardless of their characteristics as malignant, benign, soft tissue or solid, as well as all stages and grades of cancers including pre-metastatic and post-metastatic cancers. The tumor may also include one or more tumor cells.

In the present application, the term “BCMA positive tumor” generally refers to a tumor that is related to the expression of BCMA protein, or a tumor in which the expression of BCMA protein is significantly increased. The BCMA positive tumor may be a non-solid tumor. The BCMA positive tumor may be selected from the group consisting of myeloma, lymphoma and hematologic malignancy. The BCMA positive tumor may include one or more tumor cells. In some cases, the BCMA positive tumor may be multiple myeloma and lymphocytoma. For example, the tumor cells may include myeloma U266 cells, B lymphocytoma Ramos cells, multiple myeloma MM.1S cells, myeloma NCI-H929 cells, myeloma OPM-2 cells and multiple myeloma RPMI 8226 cells.

In the present application, the term “monoclonal antibody” generally refers to a population of substantially homogeneous antibodies, that is, various antibodies contained in the population are the same except potential naturally occurring mutations present in a trace amount. The monoclonal antibody can be highly specific, and directly target a single antigenic site. The monoclonal antibody can be prepared by a hybridoma technology or produced in bacteria, eukaryotic animal or plant cells by using recombinant DNA methods. The monoclonal antibody can also be obtained from a phage antibody library, by using a technology as described in, e.g., Clackson et al., Nature, 352:624-628 (1991) and Marks et al., Mol. Biol., 222:581-597 (1991).

In the present application, the term “single-chain antibody” (scFv) generally refers to a molecule including antibody heavy chain variable regions and light chain variable regions. For example, the scFv can be formed by linking an antibody heavy chain variable region to a light chain variable region via a linker (e.g., a linking peptide).

In the present application, the term “chimeric antibody” generally refers to such an antibody in which a part of the amino acid sequences of the heavy chain or the light chain is homogeneous to the corresponding amino acid sequence in an antibody derived from specific species or belongs to a certain class, while the other part of the chain is homogeneous to the corresponding sequence in another species. For example, the variable regions of the light chain and the heavy chain can be derived from the variable region of the antibody of an animal species (e.g., mice, rats, and the like), while the constant part is homogeneous to the sequence of an antibody derived from another species (e.g., human). For example, to obtain a chimeric antibody, the variable region can be produced by using non-human B cells or hybridoma cells, while the constant region combined therewith is derived from human. Since the constant region of the chimeric antibody can be derived from human, the chimeric antibody is less likely to elicit an immune response when injected than using an antibody whose constant regions are not derived from human.

In the present application, the term “humanized antibody” generally refers to an antibody that includes less sequences derived from non-human immunoglobulin, so as to reduce the immunogenicity when a heterogeneous antibody is introduced into a human. For example, it is feasible to use CDR transplant (Jones et al., Nature 321:522 (1986)) and its variants; including “reshaping” (Verhoeyen, et al., 1988 Science 239:1534-1536; Riechmann, et al., 1988 Nature 332:323-337; Tempest, et al., Bio/Technol 1991 9:266-271), “hyperchimerization” (Queen, et al., 1989 Proc Natl Acad Sci USA 86:10029-10033; Co, et al., 1991 Proc Natl Acad Sci USA 88:2869-2873; Co, et al., 1992 J Immunol 148:1149-1154) and “veneering” (Mark, et al., “Derivation of therapeutically active humanized and veneered anti-CD18 antibodies.” In: Metcalf B W, Dalton B J, eds. Cellular adhesion: molecular definition to therapeutic potential. New York: Plenum Press, 1994: 291-312), surface rendering (U.S. Pat. No. 5,639,641) and other technical means to humanize the binding domain of non-human sources. If other regions, e.g., the hinge region and the constant region domain, are also derived from non-human sources, these regions can also be humanized.

In the present application, the term “fully human antibody” generally refers to a full human antibody, that is, both the constant region and the variable region of the antibody are derived from human. The fully human antibody can be achieved by phage antibody library technology, production of a humanized antibody from transgenic mice, ribosome display technology, EBV transformed B cell cloning technology, single B cell cloning and other technologies.

In the present application, the term “multispecific antibody” generally refers to an antibody molecule capable of recognizing two or more antigens or epitopes at the same time. The multispecific antibody can be obtained in a eukaryotic expression system or in a prokaryotic expression system by chemical coupling methods, hybrid-hybridoma methods, genetically engineered antibody preparation methods and other methods.

In the present application, the term “immunoconjugate” generally refers to a conjugate formed by linking the additional reagents (e.g., chemotherapeutic agents, radioactive elements, cell growth inhibitors and cytotoxic agents) with the antibody or the antigen-binding fragment thereof. The linkage can be through covalent bonds, or non-covalent interactions, and can include chelation. The conjugate can deliver the additional reagents to the target cells (e.g., tumor cells) by the specific binding of the antibody or the antigen-binding fragment thereof to the antigen on the target cells. Then, the immunoconjugate is internalized, and finally enters into the target cells (e.g., into vesicles such as lysosome, etc.), and at that time the linking molecule in the immunoconjugate can be cleaved to release the additional reagents, thereby exerting its cytotoxic effect. Moreover, the antigen can also be secreted by the target cells, and located in a gap outside the target cells. The immunoconjugate can be formed from a variety of linking molecules known in the art. In the present application, the term “linking molecule” generally refers to a functional molecule linking or connecting two molecules. For example, the linking molecule can link one molecule to another molecule (e.g., one molecule is a protein molecule, and another molecule is also a protein molecule, or can be a small molecular drug). The linking molecule can be used in the construction of the immunoconjugate. In the immunoconjugate, the linking molecule can have two functional characteristics: 1) The linking molecule is stable in the circulating system, that is, the linking molecule cannot be cleaved in the circulating system to release the additional reagents before the immunoconjugate reaches the target cell, so as to avoid toxic effects; 2. After the immunoconjugate enters the target cell, the linking molecule needs to be broken rapidly and effectively, so that the additional reagents can be effectively released to exert their due pharmacological activity. The linking molecule can be composed of polar or nonpolar amino acids. The linking molecule can also be a carbon chain containing heteroatom(s) (e.g., nitrogen atoms, sulfur atoms, and the like). The length of the linking molecule can be between 2 and 100 atoms, e.g., between 2 and 50 atoms, and can also be 3, 5, 10, 15, 20, 25, 30, 35, 40, 45 or 50 atoms; alternatively, e.g., the length of the linker can be 20 to 26 (20, 21, 22, 23, 24, 25 or 26) atoms. The linking molecule can be substituted by a substituent selected from the group consisting of hydrogen atoms, alkyl, alkenyl, alkynyl, amino, alkylamino, dialkylamino, trialkylamino, hydroxyl, alkoxy, halogen, aryl, heterocyclyl, aromatic heterocyclyl, cyano, amide, carbamoyl, carboxylic acid, ester, thioether, alkylthioether, mercapto, and ureido. Moreover, the linking molecule can be selected from the group consisting of pH-sensitive linking molecule, proteinase-cleavable linking molecule, nuclease-sensitive linking molecule, lipase-sensitive linking molecule, glycosidase linking molecule, hypoxic linking molecule, photocleavable linking molecule, thermally unstable linking molecule and ultrasound-sensitive linking molecule, peptide linker. Exemplary linking molecules can include, but not limited to, 1,4-bis(maleimide)butane, (1,4-bismaleimido-2,3-dihydroxybutane), bis(maleimide)hexane, bis(maleimide)ethane, 1,4-di-[3′-(2′-pyridyldithio)propionamido]butane, 1,6-hexane-bis-vinyl sulfone, dithiobismaleimide ethane, 1,8-bismaleimido-diethylene glycol and 1,11-bismaleimido-triethylene glycol. For example, it may be succinimidyl 4-(N-maleimidomethyl)cyclohexane-1-carboxylate (SMCC). Common peptide linkers are well known in the art (see, for example, Holliger et al. (1993) Proc. Natl. Acad. Sci. USA 90:6444-6448; Poljak et al. (1994) Structure 2:1121-1123). For example, the linking molecule may be succinimidyl 4-(N-maleimidomethyl)cyclohexane-1-carboxylate (SMCC).

In the present application, the term “covalent” generally refers to covalent bonding, that is, two or more atoms share a common electron pair and reach a state of electron saturation to form a relatively stable chemical structure. The formation of a covalent bond involves the pairing of electrons with opposite spin directions between two adjacent atoms. At this time, the atomic orbits overlap each other, and the electron cloud density between the two nuclei increases relatively, thereby increasing the attraction to the two nuclei. Covalent bonds can have saturation and directionality. Covalent bonds can be divided into non-polar covalent bonds, polar covalent bonds and coordination bonds. Compounds containing only covalent bonds can be called covalent compounds.

In the present application, the term “chemotherapeutic agent” generally refers to an agent for chemotherapy that can inhibit the proliferation of tumors and/or tumor cells. The chemotherapeutic agent may be selected from the group consisting of mitotic inhibitors, kinase inhibitors, alkylating agents, antimetabolites, embedded antibiotics, growth factor inhibitors, cell cycle inhibitors, enzymes, topoisomerase inhibitors, histone deacetylase inhibitors, anti-survival agents, biological response modifiers, anti-honnones such as anti-androgens and anti-angiogenic agents. For example, the chemotherapeutic agent may be selected from the group consisting of capecitabine, daunomycin, daunorubicin, actinomycin D, doxorubicin, epirubicin, idarubicin, esorubicin, bleomycin, mafosfamide, ifosfamide, cytarabine, dichloroethyl nitrosourea, busulfan, mitomycin C, actinomycin D, plicamycin, prednisone, hydroxyl progesterone, testosterone, tamoxifen, dacarbazine, procarbazine, hexamethylmelamine, pentamethylmelamine, mitoxantrone, amsacrine, chlorambucil, methyl cyclohexylnitrosourea, nitrogen mustard, melphalan, cyclophosphamide, 6-mercaptopurine, 6-thioguanine, cytarabine (CA), 5-azacytidine, hydroxyurea, pentostatin, 4-hydroxy peroxycyclophosphamide, 5-fluorouracil (5-FU), 5-fluorodeoxyuridine (5-FUdR), methotrexate (MTX), colchicine, taxol, vincristine, vinblastine, etoposide, trimetrexate, teniposide and/or diethylstilbestrol (DES).

In the present application, the term “radioactive element” generally refers to an element for radiotherapy that can inhibit the proliferation of tumors and/or tumor cells. The radioactive elements can be selected from the group consisting of ³H, ¹⁴C, ¹⁵N, ³⁵S, ⁹⁰Y, ⁹⁹Tc, ¹¹¹In, ¹²⁵I and/or ¹³¹I.

In the present application, the term “cell growth inhibitor” generally refers to an agent that inhibits tumors by inhibiting growth factors that promote the growth and replication of tumor cells. The growth factors bind to the receptors on the cell surface to activate the intracellular signaling pathways. Complicated pathways may contribute to uncontrolled cell growth, leading to excessive cell division and tumor development. The cell growth inhibitor can inhibit the effects of these growth factors. The cell growth inhibitor can be selected from the group consisting of angiogenesis inhibitory factors, deacetylase (HDAC) inhibitory factors, Hedgehog signaling pathway blockers, mTOR inhibitors, p53/mdm2 inhibitors, PARP inhibitors, proteasome inhibitors and/or tyrosine kinase inhibitors.

In the present application, the term “cytotoxic agent” generally refers to an agent that inhibits the proliferation of tumors and/or tumor cells by producing toxin on the cells on which it acts. The cytotoxic agent can be selected from the group consisting of alkylating agents such as, busulfan, hexamethylmelamine, thiotepa, cyclophosphamide, nitrogen mustard, uramustine, melphalan, chlorambucil, carmustine, streptozotocin, dacarbazine, temozolomide, ifosfamide, etc.; antitumor agents, e.g., mitomycin C, etc.; antimetabolites, e.g., methotrexate, azathioprine, mercaptopurine, fludarabine, 5-fluorouracil, etc.; platinum-containing anticancer agents, e.g., cisplatin, carboplatin, etc.; anthracyclines, e.g., daunorubicin, doxorubicin, epirubicin, idarubicin, mitoxantrone, etc.; plant alkaloids and terpenoids, e.g., vincristine, vinblastine, vinorelbine, vindesine, podophyllotoxin, docetaxel, etc.; topoisomerase inhibitors, e.g., irinotecan, amsacrine, topotecan, etoposide, teniposide, etc.; antibodies, e.g., rituximab, trastuzumab, bevacizumab, erlotinib, dactinomycin, etc.; finasteride; aromatase inhibitors; tamoxifen; goserelin; paclitaxel and/or imatinib mesylate. The cytotoxic agent can be administered by oral administration, injection, and the like.

In the present application, the term “maytansine” generally refers to a compound isolated from plants of Meldenia spp. (see U.S. Pat. No. 3,896,111), which belongs to a kind of anti-mitotic cytotoxin, its structural formula is as follows:

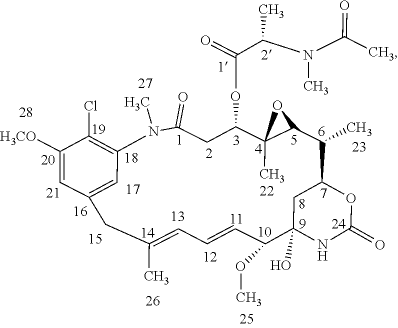

The CAS No. of the maytansine is 35846-53-8. Maytansine may have significant therapeutic effects on a variety of tumors, such as L-1210, P-388 leukemia, S-180, W-256, Lewis lung cancer and in vitro nasopharyngeal carcinoma. The maytansine derivatives may include compounds having the ring structure of maytansine and modified with one or more substituents on the ring, e.g., maytansine derivatives DM1, DM4.

In the present application, the term “maytansine derivative DM1” generally refers to a compound having a structural formula as follows.

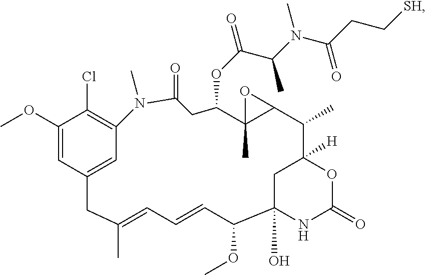 [SMILES: COc1cc2cc(c1Cl)N(C)C(=O)C[C@H](OC(=O)[C@H](C)N(C)C(=O)CCS)[C@]1(C)OC1[C@H](C)[C@@H]1C[C@@](O)(NC(=O)O1)[C@H](OC)/C=C/C=C(\C)C2]

Its CAS No. is 139504-50-0. The maytansine derivative DM1 may be a kind of anti-mitotic cytotoxin.

In the present application, the term “diseases or conditions related to the expression of BCMA protein” generally refers to diseases or conditions related to the expression of BCMA protein, or caused by the up-regulation of the expression of BCMA protein. The diseases or conditions related to the expression of BCMA protein may be proliferative diseases, precancerous conditions, malignant tumors, atypical cancers and non-cancer related indications related to the expression of BCMA protein. For example, the diseases or conditions related to the expression of BCMA protein may include non-solid tumors. For example, the diseases or conditions may be selected from the group consisting of myeloma, lymphoma and hematologic malignancy.

In the present application, the term “nucleic acid molecule” generally refers to isolated nucleotides, deoxyribonucleotides or ribonucleotides of any length, or analogues thereof isolated from its natural environment or synthesized artificially.

In the present application, the term “vector” generally refers to a nucleic acid molecule capable of self-replication in a suitable host, which transfers the inserted nucleic acid molecule into and/or between host cells. The vector may include a vector mainly used for inserting DNA or RNA into cells, a vector mainly used for replicating DNA or RNA, and a vector mainly used for expression of DNA or RNA transcription and/or translation. The vector also includes a vector with a variety of the above functions. The vector may be a polynucleotide that can be transcribed and translated into a polypeptide when introduced into a suitable host cell. Generally, by culturing a suitable host cell comprising the vector, the vector can produce the desired expression products.

In the present application, the term “cell” generally refers to an individual cell, cell line, or cell culture that can include or has included a plasmid or vector comprising the nucleic acid molecule of the present application, or can express the antibody or the antigen-binding fragment thererof of the present application. The cell can include the progeny of a single cell. Due to natural, accidental or intentional mutations, the progeny cell may not necessarily be exactly the same as the original parent cells in terms of morphology or genome, as long as it can express the antibody or the antigen-binding fragment thereof of the present application. The cell can be obtained by transfecting cells in vitro with the vector of the present application. The cell can be a prokaryotic cell (e.g., Escherichia coli) or a eukaryotic cell (e.g., yeast cells, e.g., COS cells, Chinese Hamster Ovary (CHO) cells, HeLa cells, HEK293 cells, COS-1 cells, NS0 cells, or myeloma cells). In some embodiments, the cell is a mammalian cell. For example, the mammalian cell can be a CHO-K1 cell. In the present application, the term “recombinant cell” generally refers to a cell into which a recombinant expression vector is introduced. The recombinant cell includes not only a certain specific cell, but also the progeny thereof.

In the present application, the term “about” generally refers to varying in a range of 0.5%-10% above or below a specified value, for example, varying in a range of 0.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, or 10% above or below a specified value.

In the present application, the term “include” generally refers to the meaning of include, encompass, contain or embrace. In some cases, it also indicates the meaning of “is”, or “composed of . . . ”.

Antibody, Antibody Binding Fragment or a Variant Thereof

In one aspect, the present application provides an antibody or an antigen-binding fragment or a variant thereof, which binds to BCMA protein at a KD value of 1.8×10⁻⁹ M or below (e.g., the KD value is not higher than about 1.8×10⁻⁹ M, not higher than about 1.5×10⁻⁹ M, not higher than about 1×10⁻⁹ M, not higher than about 5×10⁻¹⁰ M, not higher than about 4×10⁻¹⁰ M, not higher than about 3×10⁻¹⁰ M, not higher than about 2×10⁻¹⁰ M, not higher than 1.6×10⁻¹⁰ M, not higher than 1.5×10⁻¹⁰ M, not higher than 1.4×10⁻¹⁴ M, not higher than 1.3×10⁻¹⁰ M, not higher than 1.2×10⁻¹⁰ M, not higher than 1.1×10⁻¹⁰ M, not higher than about 1×10⁻¹⁰ M or not higher than about 1×10⁻¹¹ M).

The antibody or the antigen-binding fragment thereof of the present application can specifically recognize a BCMA protein expressed on cell surfaces.

The antibody or the antigen-binding fragment thereof of the present application can be internalized by cells. For example, the internalization may include the following steps: when the antibody or the antigen-binding fragment thereof can bind to the plasma membrane of the cells (e.g., tumor cells), or alternatively, can be released in the cells in response to the proteolytic activity in the cellular microenvironment (e.g., tumor cell microenvironment), the antibody or the antigen-binding fragment thereof can thereby be engulfed by the cell membrane and drawn into the cells. In some embodiments, the antibody or the antigen-binding fragment thereof in the immunoconjugate, and/or additional reagents conjugated therewith can also be engulfed by the cell membrane and drawn into the cells after the antibody or the antigen-binding fragment thereof has bound to the plasma membrane of the cells.

The BCMA protein may include variants of BCMA protein. For example, the variants may be: 1) proteins or polypeptides formed by substitution, deletion, or addition of one or more amino acids in the amino acid sequence of the BCMA protein; and 2) proteins or polypeptides with at least about 85% (e.g., at least about 85%, about 90%, about 91%, about 92%, about 93%, about 94%, about 95%, about 96%, about 97%, about 98%, about 99% or higher) of sequence homology with the BCMA protein.

In the present application, the cells may include tumor cells. For example, the tumor may be a BCMA positive tumor. The tumor may be a non-solid tumor. For example, the tumor may include a tumor selected from the group consisting of myeloma, lymphoma and hematologic malignancy.

In the present application, the cells may include human cells. For example, the cells may include human myeloma U266 cells, human B lymphocytoma Ramos cells, human multiple myeloma MM.1S cells, human myeloma NCI-H929 cells, human myeloma OPM-2 cells and human multiple myeloma RPMI 8226 cells.

The antibody of the present application may be selected from the group consisting of a monoclonal antibody, a single-chain antibody, a chimeric antibody, a multispecific antibody, a humanized antibody and a fully human antibody.

The antigen-binding fragment of the present application may be selected from the group consisting of Fab, Fab′, F(ab)₂, Fv and ScFv fragment.

The antibody or the antigen-binding fragment thereof of the present application may compete with a reference antibody for binding to the BCMA protein. The reference antibody may include a light chain variable region and a heavy chain variable region. For example, the light chain variable region of the reference antibody may include LCDR1, LCDR2 and LCDR3, the LCDR1 includes an amino acid sequence as set forth in SEQ ID NO:1; the LCDR2 includes an amino acid sequence as set forth in SEQ ID NO: 2; the LCDR3 includes an amino acid sequence as set forth in SEQ ID NO: 3. The heavy chain variable region of the reference antibody may include HCDR1, HCDR2 and HCDR3, the HCDR1 includes an amino acid sequence as set forth in SEQ ID NO: 4; the HCDR2 includes an amino acid sequence as set forth in SEQ ID NO: 5; the HCDR3 includes an amino acid sequence as set forth in SEQ ID NO: 6.

In the present application, the competitive binding capacity can be evaluated by determining the dissociation equilibrium constant of the antibody-antigen interaction of the antibody or the antigen-binding fragment thereof. The method of detecting the dissociation equilibrium constant can be selected from the group consisting of Enzyme-linked immunosorbent assay, surface plasmon resonance (SRP) method, potentiometric titration, spectrophotometry, capillary electrophoresis, fluorometry and thin-layer chromatography pH method. For example, the antibody or the antigen-binding fragment thereof can be detected by the SRP method (e.g., using a biomacromolecular interaction meter). It is found through detection that the antibody, the binding fragment or variant thereof of the present application can bind to the BCMA protein at a KD value of 1.8×10⁻⁹ M or below.

For example, the light chain variable region of the reference antibody may include an amino acid sequence as set forth in SEQ ID NO: 7, and the heavy chain variable region of the reference antibody may include an amino acid sequence as set forth in SEQ ID NO: 8. For another example, the amino acid sequence of the light chain of the reference antibody may include an amino acid sequence as set forth in SEQ ID NO: 9; and the heavy chain of the reference antibody may include an amino acid sequence as set forth in SEQ ID NO: 10.

For example, the antibody or the antigen-binding fragment thereof of the present application competes with a reference antibody for binding to the BCMA protein. The reference antibody may include LCDR1-3 and HCDR1-3, and LCDR1 may include an amino acid sequence as set forth in SEQ ID NO: 1; LCDR2 may include an amino acid sequence as set forth in SEQ ID NO: 2; LCDR3 may include an amino acid sequence as set forth in SEQ ID NO: 3; HCDR1 may include an amino acid sequence as set forth in SEQ ID NO: 4; HCDR2 may include an amino acid sequence as set forth in SEQ ID NO: 5; HCDR3 may include an amino acid sequence as set forth in SEQ ID NO: 6. In some embodiments, the reference antibody may include the antibody SG1116 or an antibody having the same LCDR1-3 and HCDR1-3 therewith. For another example, the reference antibody may include a light chain variable region and a heavy chain variable region, the amino acid sequence of the light chain variable region may include an amino acid sequence as set forth in SEQ ID NO: 7; and the heavy chain variable region may include an amino acid sequence as set forth in SEQ ID NO: 8. In some embodiments, the antibody or the antigen-binding fragment thereof may include the antibody SG1116 or an antibody having the same light chain variable region and heavy chain variable region therewith. For another example, the reference antibody may include a light chain and a heavy chain, the amino acid sequence of the light chain may include an amino acid sequence as set forth in SEQ ID NO: 9; and the amino acid sequence of the heavy chain may include an amino acid sequence as set forth in SEQ ID NO: 10. In some embodiments, the antibody or the antigen-binding fragment thereof may include the antibody SG1116 or an antibody having the same light chain variable region and heavy chain variable region therewith.

In the present application, the antibody or the antigen-binding fragment thereof has binding specificity. For example, the antibody or the antigen-binding fragment thereof specifically recognizes antigen, i.e., the BCMA protein, but does not recognize other proteins. For example, the other proteins may be selected from the group consisting of protein mixtures derived from animals, CD family of immune cell surface antigen molecules, tumor markers and TAM receptors, etc. For example, the other proteins may include: milk, BSA, CD19, TROP2, AXL, CD47, CD38 and Gas6. In the present application, the binding specificity of the antibody or the antigen-binding fragment thereof can be detected by the ELISA method. For example, the absorbance OD value of the antibody or the antigen-binding fragment thereof of the present application to the BCMA protein at a wavelength of 450 nm may be greater than 2.5, while the absorbance OD value to the other proteins at the same wavelength is 0.5 or less.

The antibody or the antigen-binding fragment thereof of the present application may include an antibody light chain or a fragment thereof.

For example, the antibody light chain or the fragment thereof may include LCDR1, and the LCDR1 may include an amino acid sequence as set forth in SEQ ID NO: 1. The antibody light chain or the fragment thereof may include LCDR2, and the LCDR2 may include an amino acid sequence as set forth in SEQ ID NO: 2. The antibody light chain or the fragment thereof may include LCDR3, and the LCDR3 may include an amino acid sequence as set forth in SEQ ID NO: 3.

The light chain of the antibody of the present application or the fragment thereof may include a light chain variable region VL, and the amino acid sequence of the light chain variable region VL may include an amino acid sequence as set forth in SEQ ID NO: 7.

For example, the antibody light chain or the fragment thereof may include an Igκ constant region, for example it may include a human Igκ constant region. For example, the human Igκ constant region may include an amino acid sequence as set forth in SEQ ID NO: 11.

In some embodiments, the amino acid sequence of the antibody light chain or the fragment thereof may include an amino acid sequence as set forth in SEQ ID NO: 9.

The antibody or the antigen-binding fragment thereof of the present application may include an antibody heavy chain or a fragment thereof.

For example, the antibody heavy chain or the fragment thereof may include HCDR1, and the HCDR1 may include an amino acid sequence as set forth in SEQ ID NO: 4. The antibody heavy chain or the fragment thereof may include HCDR2, and the HCDR2 may include an amino acid sequence as set forth in SEQ ID NO: 5. For another example, the antibody heavy chain or the fragment thereof may include HCDR3, and the HCDR3 may include an amino acid sequence as set forth in SEQ ID NO: 6.

The antibody heavy chain or the fragment thereof may include a heavy chain variable region VH, and the heavy chain variable region VH may include an amino acid sequence as set forth in SEQ ID NO: 8.

For example, the antibody heavy chain or the fragment thereof also includes a human constant region. Where, the human constant region may include a human IgG constant region. For example, the human IgG constant region may include a human IgG1 constant region. For example, the human IgG1 constant region may include an amino acid sequence as set forth in SEQ ID NO: 12.

In some embodiments, the amino acid sequence of the antibody heavy chain may include an amino acid sequence as set forth in SEQ ID NO: 10.

In some embodiments, in the antibody or the antigen-binding fragment thereof of the present application, LCDR1 may include an amino acid sequence as set forth in SEQ ID NO: 1; LCDR2 may include an amino acid sequence as set forth in SEQ ID NO: 2; LCDR3 may include an amino acid sequence as set forth in SEQ ID NO: 3; and HCDR1 may include an amino acid sequence as set forth in SEQ ID NO: 4; HCDR2 may include an amino acid sequence as set forth in SEQ ID NO: 5; HCDR3 may include an amino acid sequence as set forth in SEQ ID NO: 6. For example, the antibody or the antigen-binding fragment thereof may include the antibody SG1116 or an antibody having the same LCDR1-3 and HCDR1-3 therewith. In some embodiments, the light chain of the antibody or the antigen-binding fragment thereof of the present application may include a light chain variable region, the light chain variable region may include an amino acid sequence as set forth in SEQ ID NO: 7; and wherein the heavy chain may include a heavy chain variable region, the heavy chain variable region may include an amino acid sequence as set forth in SEQ ID NO: 8. For example, the antibody or the antigen-binding fragment thereof may include the antibody SG1116 or an antibody having the same light chain variable region and heavy chain variable region therewith.

In some embodiments, the antibody of the present application may be SG1116. The amino acid sequences of LCDR1-3 of the antibody SGT116 may be as set forth in SEQ ID NO: 1, SEQ ID NO: 2 and SEQ ID NO: 3 respectively, and the amino acid sequences of HCDR1-3 may be as set forth in SEQ ID NO: 4, SEQ ID NO: 5 and SEQ ID NO: 6 respectively; the amino acid sequence of VL may be as set forth in SEQ ID NO: 7, and the amino acid sequence of VH may be as set forth in SEQ ID NO: 8.

It should also be understood that the protein, polypeptide and/or amino acid sequence involved in the present application include at least variants or homologs having the same or similar functions as the protein or polypeptide.

In the present application, the variant may be a protein or polypeptide formed by substitution, deletion or addition of one or more amino acids in the amino acid sequence of the protein and/or the polypeptide (e.g., the antibody or the antigen-binding fragment thereof of the present application). For example, the variant can include a protein or polypeptide with changes of amino acids by substitution, deletion and/or insertion of at least 1, e.g., 1-30, 1-20 or 1-10, or e.g., 1, 2, 3, 4, or 5 amino acids. The functional variant can substantially maintain the biological characteristics of the protein or polypeptide before change (e.g., substitution, deletion or addition). For example, the functional variant can maintain at least 60%, 70%, 80%, 90%, or 100% of the biological activity (e.g., the ability of specifically binding to the BCMA protein) of the protein or polypeptide before change.

In the present application, the homolog can be a protein or polypeptide having at least about 80% (e.g., at least about 85%, about 90%, about 91%, about 92%, about 93%, about 94%, about 95%, about 96%, about 97%, about 98%, about 99% or higher) of sequence homology with the amino acid sequence of the protein and/or the polypeptide (e.g., the antibody or the antigen-binding fragment thereof of the present application).

In the present application, the homology generally refers to the degree of similarity or relevance among two or more sequences. The “percentage of sequence homology” can be calculated by the following way: the two sequences to be aligned are compared in a comparison window to determine the number of positions having the same nucleic acid bases (e.g., A, T, C, G, I) or the same amino acid residues (e.g., Ala, Pro, Ser, Thr, Gly, Val, Leu, Ile, Phe, Tyr, Trp, Lys, Arg, His, Asp, Glu, Asn, Gln, Cys and Met) in the two sequences to obtain the number of matching positions. The number of matching positions is divided by the total position number in the comparison window (i.e., the window size), and the result is multiplied by 100 to give the percentage of sequence homology. The alignment for determining the percentage of sequence homology can be performed in accordance with various methods known in the art, e.g., by use of publicly available computer softwares, e.g., BLAST, BLAST-2, ALIGN or Megalign (DNASTAR) softwares. Persons skilled in the art can determine appropriate parameters for the sequence alignment, including any algorithm required to achieve the maximum alignment within the full-length sequence being compared or within the target sequence region. The homology can also be determined by the following methods: FASTA and BLAST. For a description of the FASTA algorithm, see “Improved Tools for Biological Sequence Comparison” to W. R. Pearson and D. J. Lipman, Proc. Natl. Acad. Sci., 85: 2444-2448, 1988; and “Fast and Sensitive Protein Similarity Search” to D. J. Lipman and W. R. Pearson, Science, 227: 1435-1441, 1989. For a description of the BLAST algorithm, see “a basic alignment search tool” to S. Altschul, W. Gish, W. Miller, E. W. Myers and D. Lipman, Journal of Molecular Biology, 215: 403-410, 1990.

Nucleic Acid, Vectors, Host Cells and Preparation Method

In another aspect, the present application also provides one or more isolated nucleic acid molecules. The one or more nucleic acid molecules can encode the antibody or the antigen-binding fragment thereof of the present application. For example, each of the one or more nucleic acid molecules can encode the complete antibody or the antigen-binding fragment thereof, and it can also encode a part thereof (e.g., one or more of HCDR1-3, LCDR1-3, VL, VH, light chain or heavy chain).

The nucleic acid molecules of the present application can be isolated from each other, e.g., they can include nucleotide sequences encoding the antibody or the antigen-binding fragment thereof of the present application or a part thereof, respectively. The nucleic acid molecules of the present application can also include a plurality of nucleotide sequences encoding the antibody or the antigen-binding fragment thereof of the present application or a part thereof simultaneously.

In the present application, the nucleic acid molecules can be synthesized by conventional methods in the art. For example, they can be produced or synthesized by the following methods: (i) amplification in vitro, e.g., being produced by amplification through polymerase chain reaction (PCR), (ii) being produced by cloning and recombination, (iii) being purified, for example fractionation by enzymatic digestion and gel electrophoresis, or, (iv) being synthesized, for example through chemical synthesis. In some cases, the nucleic acid molecules can be prepared by a recombinant DNA technology.

In the present application, the nucleic acid encoding the antibody or the antigen-binding fragment thereof can be prepared by various methods known in the art, including, but not limited to, overlap extension PCR using restrictive fragment operation or using synthetic oligonucleotide. For specific operations, see Sambrook et al., Molecular Cloning, A Laboratory Manual, Cold Spring Harbor Laboratory Press, Cold Spring Harbor, N.Y., 1989; and Ausube et al. Current Protocols in Molecular Biology, Greene Publishing and Wiley-Interscience, New York N.Y., 1993.

In another aspect, the present application provides one or more vectors, comprising the one or more nucleic acid molecules of the present application. Each vector can include one or more of the nucleic acid molecules. Moreover, the vector can further include other gene(s), e.g., a marker gene that allows the selection of the vector in an appropriate host cell and under appropriate conditions. Moreover, the vector can further include an expression control element that allows the coding region to be properly expressed in an appropriate host. Such a control element is well known by persons skilled in the art. For example, it can include a promoter, a ribosome binding site, an enhancer and other control elements regulating the gene transcription or the mRNA translation, and the like. The one or more nucleic acid molecules of the present application can be operatively linked to the expression control element.

The vector can include, e.g., plasmid, cosmid, virus, phage, or other vectors commonly used in, e.g., genetic engineering. For example, the vector is an expression vector.

In another aspect, the present application provides cells, which may include the one or more nucleic acid molecules of the present application and/or the one or more vectors of the present application. For example, each cell may include one nucleic acid molecule or one vector of the present application. For example, each cell may include a plurality of (e.g., two or more) nucleic acid molecules or vectors of the present application. For example, the vectors of the present application can be introduced into the cells, e.g., eukaryotic cells, such as cells from plants, fungi or yeast cells, and the like. The vectors of the present application can be introduced into the cells by methods known in the art, such as electroporation, lipofectine transfection, lipofectamin transfection, and the like. For example, the cells may be CHO-S.

In another aspect, the present application provides a method of preparing the antibody or the antigen-binding fragment thereof. The method may include culturing the cells of the present application under conditions allowing the expression of the antibody or the antigen-binding fragment thereof. For example, the method can be performed by employing appropriate medium, appropriate temperature and culturing time, and the like, which are known to persons of ordinary skills in the art. The present application provides a method of preparing the antibody or antigen-binding fragment using the method of phage display library, comprising the steps as follows: synthesizing a library of human antibodies on the phage, screening the library with BCMA or a part thereof, isolating phages that bind to BCMA; infecting bacteria with phages, amplifying and culturing and then selecting monoclones that specifically bind to antigen; sequencing to get the sequences of the heavy chain variable region and the light chain variable region encoding the antibody; cloning the variable region gene into a eukaryotic expression vector, e.g., pCMV-163, and transfecting into host cells for expression of the antibody.

In some cases, the method may also include steps of isolating and/or purifying the antibody or the antigen-binding fragment thereof. For example, the antibody or the antigen-binding fragment thereof of the present application can be purified and isolated by affinity chromatography using protein G-sepharose or protein A-sepharose, or by gel electrophoresis and/or high performance liquid chromatography. For example, the protein A affinity purification can also be used.

Immunoconjugate, Pharmaceutical Composition, Application

In another aspect, the present application provides an immunoconjugate, comprising the antibody or the antigen-binding fragment thereof.

For example, the immunoconjugate may include at least one additional reagent selected from the group consisting of a chemotherapeutic agent, a radioactive element, a cell growth inhibitor and a cytotoxic agent. In some embodiments, the antibody or the antigen-binding fragment thereof in the immunoconjugate may be directly or indirectly linked to the at least one additional reagent. For example, the antibody or the antigen-binding fragment thereof in the immunoconjugate may be linked to the at least one additional reagent through a linking molecule, e.g., through an SMCC (succinimidyl 4-(N-maleimidomethyl)cyclohexane-1-carboxylate) linker.

In the present application, the at least one additional reagent may include maytansine or a derivative thereof. For example, the maytansine derivative may include maytansine derivative DM1.

In another aspect, the present application provides a pharmaceutical composition, comprising the antibody or the antigen-binding fragment thereof, the immunoconjugate, or the cells, as well as optionally a pharmaceutically acceptable adjuvant.

The pharmaceutically acceptable adjuvant may include buffers, antioxidants, preservatives, low molecular weight polypeptides, proteins, hydrophilic polymers, amino acids, carbohydrates, chelating agents, counterions, metal complexes and/or nonionic surfactants, etc.

In the present application, the pharmaceutical composition can be formulated for oral administration, intravenous administration, intramuscular administration, in situ administration at the tumor site, inhalation, rectal administration, vaginal administration, transdermal administration or administration via subcutaneous reservoir. For example, for injection preparations, the pharmaceutical composition can be prepared into, for example, a single-dose dosage form of ampoules or, for example, a unit dosage form of multi-dose container. The pharmaceutical composition can also be prepared into solutions, suspensions, tablets, pills, capsules and long-acting preparations.

The pharmaceutical composition can be used for inhibiting the growth of tumors (e.g., BCMA positive tumor). For example, the pharmaceutical composition of the present application can inhibit or delay the development or progression of a disease, reduce the tumor size (or even substantially eliminate the tumor), and/or alleviate and/or stabilize the disease state.

The pharmaceutical composition of the present application may include a therapeutically effective amount of the antibody or the antigen-binding fragment thereof. The therapeutically effective amount is a dose required to prevent and/or treat (at least partially treat) a disease or disorder (e.g., cancer) and/or any complications thereof in a subject suffering from or having a developing risk of the disease or disorder.

In another aspect, the present application provides a use of the antibody or the antigen-binding fragment thereof, or the immunoconjugate in the preparation of a medicament, the medicament is used for preventing or treating a tumor.

In the present application, the tumor may include a BCMA positive tumor. In some embodiments, the tumor may include a non-solid tumor. In some cases, the tumor may be B cell cancers, multiple myeloma, malignant plasma cell tumor, Kahler's disease and myeloma; plasma cell leukemia; plasmocytoma; B cell prolymphocytic leukemia; hairy cell leukemia; B cell non-Hodgkin lymphoma (NHL); acute myeloid leukemia (AML); chronic myeloid leukemia (CML); acute lymphocytic leukemia (ALL); chronic lymphocytic leukemia (CLL); follicular lymphoma (including the type of follicular non-Hodgkin lymphoma); Burkitt's lymphoma (endemic Burkitt's lymphoma; sporadic Burkitt's lymphoma); marginal zone lymphoma (mucosa-associated lymphoid tissue; MALT/MALToma; monocytic B cell lymphoma; splenic lymphoma with villous lymphocytes); mantle cell lymphoma; large cell lymphoma (diffuse large cell; diffuse mixed-cell; immunoblastic lymphoma; primary mediastinal B cell lymphoma; angiocentric lymphoma-lung B cells); small lymphocytic lymphoma (SLL); precursor B-lymphoblastic lymphoma; myeloid leukemia (granulocytic; myeloid; acute myeloid leukemia; chronic myeloid leukemia; subacute myeloid leukemia; myeloid sarcoma; Green tumor; granulocytic sarcoma; acute promyelocytic leukemia; acute myelomonocytic leukemia); Waldenstrom macroglobulinemia or other B cell lymphoma. For example, the tumor may include a tumor selected from the group consisting of myeloma, lymphoma and hematologic malignancy.

In the present application, the term “lymphoma” generally refers to blood cell tumors caused by lymphocytic lesions. Lymphoma cell surfaces are usually positive for BCMA expression.

In the present application, the term “myeloma” generally refers to tumors characterized by clonal proliferation of plasma cells in bone marrow. On myeloma cells, the expression of BCMA is usually positive. Exemplary myeloma cells include, but not limited to, X63Ag8, Sp2/0, NS1, NS0, J558L, U266, U937, P3U1, XG-1, XG-2, XG-3, XG-4, XG-5, XG-6, XG-7, XG-8, XG-9, U266, RPM1-8226, LP1, L363, OPM1, OPM2, as well as NCLH929 cells or cell lines derived from these cell lines.

In the present application, the term “hematologic malignancy” generally refers to cancers or hyperproliferative disorders produced during the hematopoiesis process that involves cells such as white blood cells, lymphocytes, natural killer cells, plasma cells and myeloid cells such as neutrophils and monocytes. Hematologic malignancies may include lymphoma, leukemia, myeloma or lymphoid malignancies as well as spleen and lymph node cancers. This term also encompasses other hematologic and/or B cell or T cell related cancers, but does not include the aforementioned multiple myeloma and lymphoma.

In another aspect, the present application provides the antibody or the antigen-binding fragment thereof, or the immunoconjugate, which is used for preventing or treating a tumor.

In the present application, the tumor may include a BCMA positive tumor. In some embodiments, the tumor may include a non-solid tumor. For example, the tumor may include a tumor selected from the group consisting of myeloma, lymphoma and hematologic malignancy.

In another aspect, the present application provides a method of preventing or treating a tumor in a subject in need thereof, which includes administering to the subject the antibody or the antigen-binding fragment thereof, the immunoconjugate, or the pharmaceutical composition.

In the present application, the tumor may include a BCMA positive tumor. In some embodiments, the tumor may include a non-solid tumor. For example, the tumor may include a tumor selected from the group consisting of myeloma, lymphoma and hematologic malignancy.

In another aspect, the present application provides the antibody or the antigen-binding fragment thereof, which is used for diagnosing diseases or conditions related to the expression of the BCMA protein.

In the present application, the diseases or conditions related to the expression of BCMA protein may be selected from the group consisting of myeloma, lymphoma and hematologic malignancy.

In another aspect, the present application provides a use of the antibody or the antigen-binding fragment thereof in the preparation of a diagnostic agent, in which the diagnostic agent is used for diagnosing diseases or conditions related to the expression of the BCMA protein.

In the present application, the diagnostic agent can be used alone or in combination with an instrument, appliance, device or system. In the processes of disease prevention, diagnosis, treatment monitoring, prognostic observation, health status evaluation, and genetic disease prediction, the diagnostic agent can be used to perform in vitro detection on human samples (for example, various body fluids, cells, tissue samples, etc.). The diagnostic agent may be selected from the group consisting of reagents, kit, calibrators and quality control products.

The method of in vitro detection may be selected from the group consisting of Western Blot, ELISA and immunohistochemistry. For example, the reagent may include reagents capable of measuring the expression of BCMA protein. For example, the reagent may be selected from the group consisting of reagents used to implement Western Blot, reagents used to implement ELISA and reagents used to implement the immunohistochemistry.

In the present application, the diseases or conditions related to the expression of BCMA protein may be selected from the group consisting of myeloma, lymphoma and hematologic malignancy.

In another aspect, the present application provides a method of diagnosing diseases or conditions related to the expression of BCMA protein in a subject, which includes: contacting a sample derived from the subject with the antibody or the antigen-binding fragment thereof, and judging the presence and/or content of substances in the sample which are capable of specifically binding to the antibody or the antigen-binding fragment thereof.

In the present application, the diseases or conditions related to the expression of BCMA protein may be selected from the group consisting of myeloma, lymphoma and hematologic malignancy.

Without intending to be limited by any theory, the following examples are only to illustrate the working modes of the device, method and system of the present application, and are not used to limit the inventive scope of the present application.

### EXAMPLES

### Example 1 Antibody Screening

Peripheral blood lymphocytes were separated from 116 healthy adult people (half for men and women) with lymphocyte separation medium. Total RNA was extracted by the Trizol method, and reversely transcribed into cDNA. A conventional PCR method was used to amplify variable region genes of different antibody subtypes, the variable region genes of antibodies were cloned into pDF vectors that have been treated with the same enzyme digestion by a conventional molecular biological technology, and the E. coli XL1-Blue was transformed by electroporation (Agilent Technology). After expanded cultivation with SB culture solution, 1×10¹³ pfu helper virus VCSM13 (BioVector NTCC Inc.) was added for infection, to obtain the primary phage antibody library. The primary phage antibody library was mixed with BS1365 bacteria (BioVector NTCC Inc.) at a certain ratio (multiplicity of infection (MOI)>200). The recombination of loxp/loxp511 was mediated by means of the Cre recombinase expressed by BS1365 bacteria, so as to construct a large-capacity antibody library.

After blocking an immune test tube coated with the recombinant BCMA-His (ACRO Biosystems) with 5% of skimmed milk powder, the above phage antibody library was added and incubated at 37° C. for 2 h. After discarding the unbound phages, the phage antibody library was washed with TBS-T lotion for 5 times to completely wash away the non-specifically adsorbed phages. The phages were eluted by adding 1 ml elution buffer (0.1 mol/L of glycine-HCl, pH=2.2) and neutralized with 40 μL of 2 mol/L Tris solution. XL1-Blue bacteria at the logarithmic period (Agilent Technology), SB medium (SB culture solution: tryptone 30 g, yeast extract 20 g, MOPS 10 g, dissolved in 950 mL deionized water, adjusting pH value to 7.0 with sodium hydroxide, metered to 1 L, high pressure sterilization) and helper phage VCSM13 were added for amplification and enrichment. The process was repeated for 3-4 cycles. The eluted phages were infected with freshly prepared logarithmic phase XL1-Blue bacteria coated culture plate. After culturing at 37° C. overnight, monoclones were randomly picked into a 96-well deep well plate (Corning), cultured expansively and then detected by phage-ELISA for the binding properties to antigens. A total of 500 clones were identified, where among the 38 clones that specifically bind to BCMA, the binding activity of SG1116 is good (for the specific method and reagents, see: Phage Display, Humana Press).

The SG1116 clone was sent for sequencing. The obtained variable region gene was analyzed online by IMGT (http://imgt.cines.fr/imgtvquest/vquest), obtaining the amino acid sequences of HCDR1-3 of the SG1116 antibody as set forth in SEQ ID NO: 4, SEQ ID NO: 5 and SEQ ID NO: 6 in turn; the amino acid sequences of LCDR1-3 as set forth in SEQ ID NO: 1, SEQ ID NO: 2 and SEQ ID NO: 3 in turn; the amino acid sequence of the light chain variable region as set forth in SEQ ID NO: 7, and the amino acid sequence of the heavy chain variable region as set forth in SEQ ID NO: 8.

### Example 2 Antibody Expression and Purification

The variable region gene of SG1116 clone obtained in Example 1 was cloned into a eukaryotic expression vector pCMV-163 containing the constant region gene of human IgG, to construct a whole antibody expression vector. The whole antibody is called SG1116 antibody.

By using ExpiCHO™ Expression System kit (purchased from Thermo Fisher Scientific), the obtained eukaryotic expression vector encoding the antibody SG1116 was transfected into CHO-S cells for expression. The cell culture supernatant containing the target protein was collected and purified by conventional Protein A affinity to get the target antibody.

### Example 3 Binding Activity of Antibody to Antigen

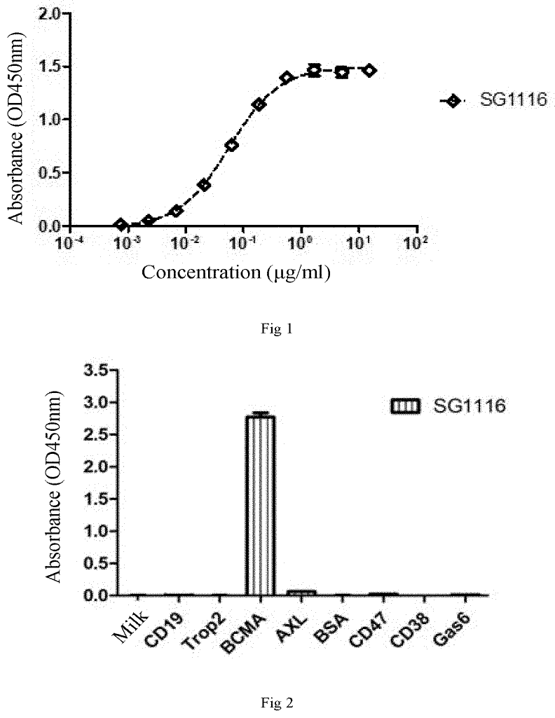

ELISA strips were coated with the target antigen BCMA-His at 1 μg/ml, overnight at 4° C. After washing with PBST, 10% of fetal calf serum was added for blocking at 37° C. for 1 hour. Different concentrations of the antibody SG1116 was added to react at 37° C. for 1 hour. After washing with PBST, horseradish peroxidase-labeled goat anti-human IgG secondary antibody (Goat anti human (IRP), thermo Fisher Scientific) was added to react at 37° C. for 30 minutes. The plate was washed with PBST repeatedly for 5 times, and patted on absorbent paper so as to dry the remaining droplets as much as possible. Each well was added with 100 d TMB (eBioscience), placed in dark at room temperature (20±5° C.) for 1.5 min. Each well was added with 100 μl of 2N H₂SO₄ stop solution to terminate the reaction of the substrate. The OD values at 450 nm were read with a Microplate reader. The binding ability of the antibody to the target antigen BCMA was analyzed. The results are shown in FIG. 1, showing that the binding activity EC₅₀ value of the antibody SG1116 to the antigen BCMA is 56.5 ng/mL.

### Example 4 Determination of Antibody Affinity

The antibody affinity was analyzed with BIACORE biomacromolecular interaction analyzer (GE Co.). Anti-human IgG antibodies (purchased from GE) were coupled on a chip, and utilized to capture the SG1116 antibody, of which the concentration was set as 3 μg/mL, and the sampling time was 120 seconds; with the antigen BCMA as the mobile phase, a gradient of 8 concentrations (0.15625, 0.3125, 0.625, 1.25, 2.5, 5, 10, 20 nM) was used, and the binding time was 120 seconds; the dissociation time was 300 seconds; each antibody was regenerated using 3 M of magnesium chloride (purchased from GE) for a time period of 30 seconds. The results show the affinity of the SG1116 antibody, as shown in Table 2.

TABLE 2
Determination of the affinity of SG1116 antibody

| | Binding | Dissociation | Dissociation equilibrium |
|---|---|---|---|
| Antibody | constant (1/Ms) | constant (1/s) | constant (M) |
| SG1116 | 1.23E+07 | 1.71E−02 | 1.39E−09 |

### Example 5 Antibody Specifically Recognizes Antigen

ELISA strips were coated with milk (Beijing Biomed Biotech Co., Ltd.), BSA (BOVOGEN), CD19 (Sino Biological Inc.), TROP2 (Sino Biological Inc.), BCMA (ACRO Biosystems), CD47 (Sino Biological Inc.), CD38 (Sino Biological Inc.), Gas6 (R&D) and various proteins as well as AXL (ACRO Biosystems), respectively, at 1 μg/ml, overnight at 4° C. After washing with PBST, 10% of fetal calf serum was added for blocking at 37° C. for 1 hour. The antibody SG1116 was added to react at 37° C. for 1 hour. After washing with PBST, horseradish peroxidase-labeled goat anti-human IgG secondary antibody (Goat anti human (HRP), Thermo Fisher Scientific) was added to react at room temperature for 30 minutes. The plate was washed with PBST repeatedly for 5 times, and patted on absorbent paper so as to dry the remaining droplets as much as possible. Each well was added with 100 μl TMB (eBioscience, #85-00-420), placed in dark at room temperature (20:5° C.) for 1.5 min. Each well was added with 100 μl of 2N H₂SO₄ stop solution to terminate the reaction of the substrate. The OD values at 450 nm were read with a Microplate reader. The binding ability of the antibody to protein was analyzed.

The results are shown in FIG. 2. It is shown from the results that the SG1116 antibody can specifically recognize the target antigen BCMA, but has no significant binding reaction with milk, CD19, TROP2, AXL, BSA, CD47, CD38, Gas6 and other proteins.

### Example 6 Expression of BCMA Antigen on Cell Surface

With human myeloma U266 cells, human B lymphocytoma Ramos cells, human multiple myeloma MM.1S cells, human myeloma NCI-H929 cells, human myeloma OPM-2 cells and human multiple myeloma RPMI 8226 cells and other polyclonal tumor cells as representative models, the expression of BCMA on the surface of tumor cells was analyzed by flow cytometry.

Cells at the logarithmic growth phase were collected, adjusted to a cell density of 5×10⁶ cells/mL, and precooled on ice. 100 μl of cells were taken and added into APC anti-human BCMA antibody (BioLegend) to react in dark at 4° C. for 20 min. At the end of the reaction, they were washed twice with pre-cooled normal saline containing 2% FBS (6000 rpm, 45 s). The cells were resuspended with 400 μl of 1% (v/v) paraformaldehyde. The expression of BCMA antigen on the cell surface was analyzed with a flow cytometer (BD Calibur).

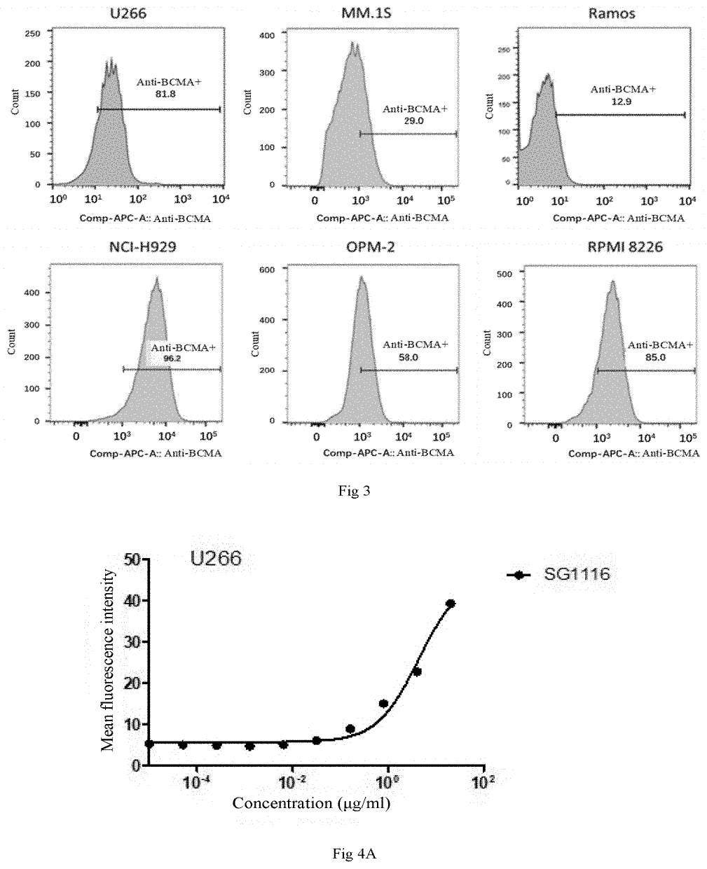

The results show that, human myeloma U266 cells, human B lymphocytoma Ramos cells, human multiple myeloma MM.1 S cells, human myeloma NCI-H929 cells, human myeloma OPM-2 cells and human multiple myeloma RPMI 8226 cells all express BCMA antigen, with the results shown in FIG. 3.

### Example 7 Antibody Specifically Recognizes Antigen on the Cell Surface

With human myeloma U266 cells as well as CHOS cells (CHOS-BCMA) stably expressing BCMA constructed by genetic engineering technology as representative cell models, the binding of BCMA on the cell surface to SG1116 was analyzed by flow cytometry. Cells at the logarithmic growth phase were collected, adjusted to a cell density of 5×10⁶ cells/mL, and precooled on ice. The SG1116 antibody was diluted with pre-cooled normal saline containing 2% FBS to 20 μg/ml. 100 μl of cells were taken and added into an equal volume of the above diluted SG1116 antibody, and reacted in dark at 4° C. for 30 min. At the end of the reaction, they were washed twice with pre-cooled normal saline containing 2% FBS (6000 rpm, 45 s). A secondary antibody PE mouse anti-human IgG (BD Pharmingen) was diluted with pre-cooled normal saline containing 2% FBS at a ratio of 1:5. 100 μl of cells were taken and resuspended, and reacted in dark at 4° C. for 30 min. At the end of the reaction, they were washed twice with pre-cooled normal saline containing 2% FBS (6000 rpm, 45 s). The cells were resuspended with 400 μl of 1% (v/v) paraformaldehyde. The binding ability of the antibody to the antigen on the cell surface was analyzed with a flow cytometer (BD Calibur).

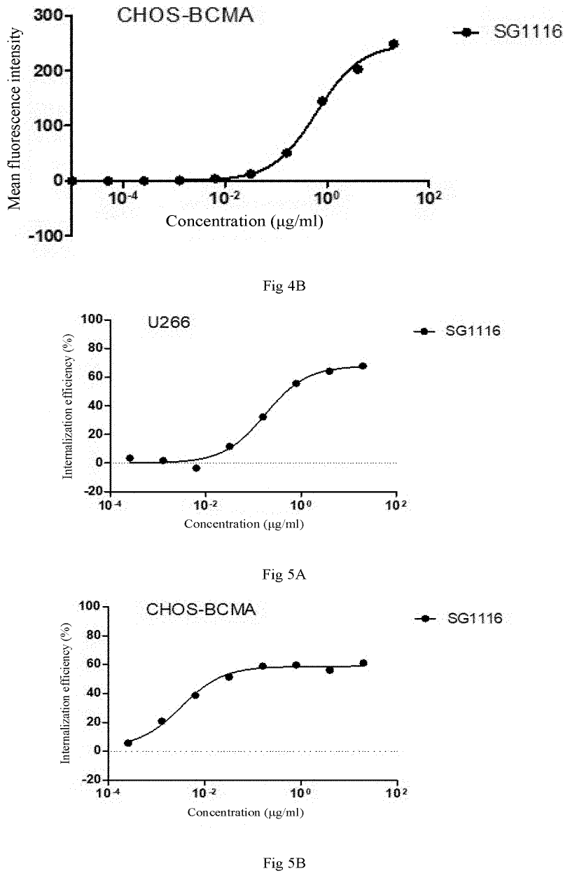

The results are shown in FIGS. 4A-4B. The results show that, the SG1116 antibody can specifically recognize the BCMA molecules on the surface of human myeloma U266 cells (FIG. 4A), and CHOS cells stably expressing BCMA constructed by genetic engineering technology (CHOS-BCMA, FIG. 4B), and the recognization activity is dose-dependent. The EC₅₀ value of binding to U266 cells is 4.294 μg/mL, and the EC₅₀ value of binding to CHOS-BCMA cells is 0.6221 μg/mL.

### Example 8 Internalization Activity of Antibody

With human myeloma U266 cells as well as CHOS cells stably expressing BCMA constructed by genetic engineering technology (CHOS-BCMA) as representative cell models, the internalization activity mediated after the SG1116 antibody recognizes BCMA was analyzed by flow cytometry. Cells at the logarithmic growth phase were collected, adjusted to a cell density of 5×10⁶ cells/mL, and precooled on ice. The SG1116 antibody was diluted with pre-cooled normal saline containing 2% FBS to different concentrations. 100 μl of cells were taken and added into an equal volume of the above diluted SG1116 antibody, and incubated at 4° C. for 30 min. At the end of the reaction, the cells were washed with pre-cooled normal saline containing 2% FBS for three times. The cells were continued to be cultured at 4° C. or 37° C. for 2 hours and then washed twice. A secondary antibody PE mouse anti-human IgG (BD Pharmingen) was diluted with pre-cooled normal saline containing 2% FBS at a ratio of 1:5. 100 μl of cells were taken and resuspended, and reacted in dark at 4° C. for 30 min. At the end of the reaction, the cells were washed for three times. The cells were resuspended with 400 μl of 1% paraformaldehyde. The fluorescence intensity on the surface of cells cultured with the antibody at different temperatures was analyzed with a flow cytometer (BD Calibur), and the internalization efficiency of the antibody was calculated following the equation as below.

Internalization efficiency=[Total MFI of the surface (4° C.)−Total MFI of the surface (37° C.)]/Total MFI of the surface (4° C.)×100%.

The results are shown in FIGS. 5A-5B. The results show that, the SG1116 antibody can be efficiently internalized on the two representative cell models of human myeloma U266 cells (FIG. 5A) and CHOS cells stably expressing BCMA (CHOS-BCMA, FIG. 5B), of which the EC₅₀ are 0.1776 μg/mL and 0.00327 μg/mL, respectively.

### Example 9 Activity of an Antibody to Inhibit Tumor In Vivo

With NCI-H929 cells as representative cells, a tumor-bearing animal model was established to evaluate the activity of SG1116 to inhibit the tumor in vivo.

Female, 6-7-week-old NOD/SCID mice (Shanghai Lingchang Biotech Co., Ltd.) were selected, and NCI-H929 cells were cultured in RPMI1640+0.05 mM β-ME culture solution containing 10% of fetal calf serum. NCI-H929 cells at the exponential growth phase were collected, resuspended in PBS to a suitable concentration and mixed with basement membrane matrix (Matrigel) at 1:1 for subcutaneous tumor inoculation on the right back of mice. After inoculation, when the average tumor volume was 54 mm³, mice were randomly divided into 2 groups according to the tumor size (a vehicle control group and an SG1116 group), with 6 mice in each group. These mice were administered by intraperitoneal injection at a dose of 5 mg/kg per dose and administered once a week for four weeks. The experiment ended one week after the last administration. When the tumor volume of a single mouse exceeded 3000 mm³, the mouse was euthanized. The tumor growth of mice was observed during administration. The efficacy was evaluated according to the relative tumor growth inhibition rate (TGI), and the safety was evaluated according to the change of animal weight and the mortality. The relative tumor growth inhibition rate TGI (%) was calculated following the equation as below: TGI %=(1−T/C)×100%. (T and C are the relative tumor volume (RTV) or tumor weight (TW) of the treatment group and the control group at a certain time point, respectively).

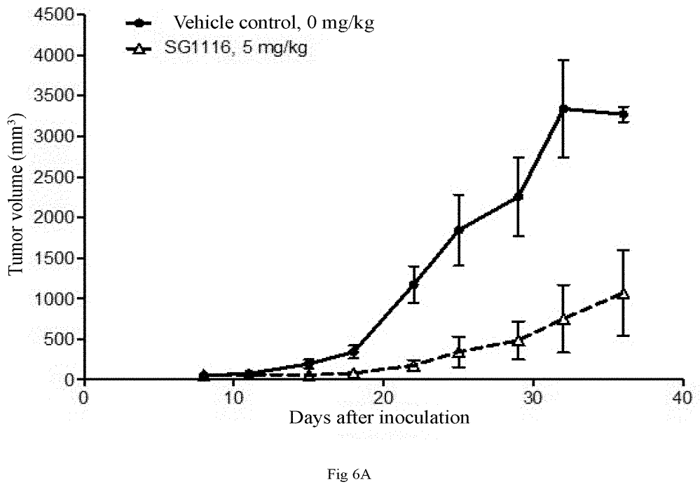

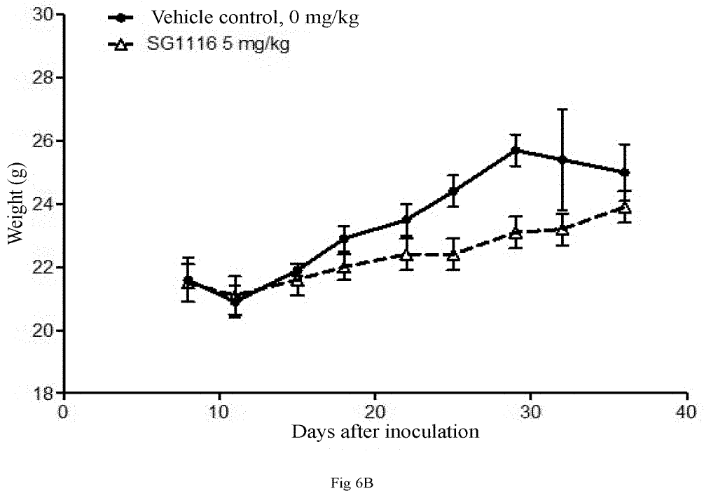

The results are shown in FIGS. 6A-6B. The group of test drug SG1116 (5 mg/kg) showed obvious anti-tumor effects on the 17th day after grouping (administration for three times), for which the relative tumor growth inhibition rate TGI (%) was 81.35%, with a statistically significant difference relative to the vehicle control group (p value <0.01).

Four mice in the vehicle control group on the 28th day after grouping were euthanized because their tumor volume exceeded 3000 mm³. The surviving mice were analyzed statistically: the SG1116 group (5 mg/kg) showed obvious anti-tumor effects, for which the relative tumor growth inhibition rate TGI (%) was 67.31%, with a statistically significant difference relative to the vehicle control group (p value <0.05).

No animals died during the treatment, no obvious drug toxicity was shown and the tolerance was good.

### Example 10 Antibody-Conjugated Small-Molecule Drug Inhibits the Proliferation of Tumor Cells

With SMCC as the linking molecule in the immunoconjugate of the present application, and with DM1 as the additional reagent in the immunoconjugate of the present application, an immunoconjugate SG1116-DM1 was constructed. With human myeloma U266 cells as representative cell models, the biological activity of the immunoconjugate was evaluated, thereby further analyzing the potential of the SG1116 antibody to construct immunoconjugates such as antibody small molecule conjugated drug (ADC).

A certain number of cells (human myeloma U266 cells) at the logarithmic growth phase were inoculated in a 96-well culture plate. After 24 hours of adherent growth, different concentrations of drugs were added to act for 72 hours. After the end of the drug action, CCK-8 (Dojindo) was added to the plate in 10 μl per well, and incubated in a 37° C., 5% CO₂ incubator for 3 to 5 hours. The OD value was determined with a Microplate reader at a wavelength of 450 nm, and the cell growth inhibition rate was calculated following the equation as below:

Inhibition rate=(OD value of the control well−OD value of the dosing well)/OD value of the control well×100%

According to the inhibition rate at each concentration, the half inhibition concentration IC₅₀ was calculated.

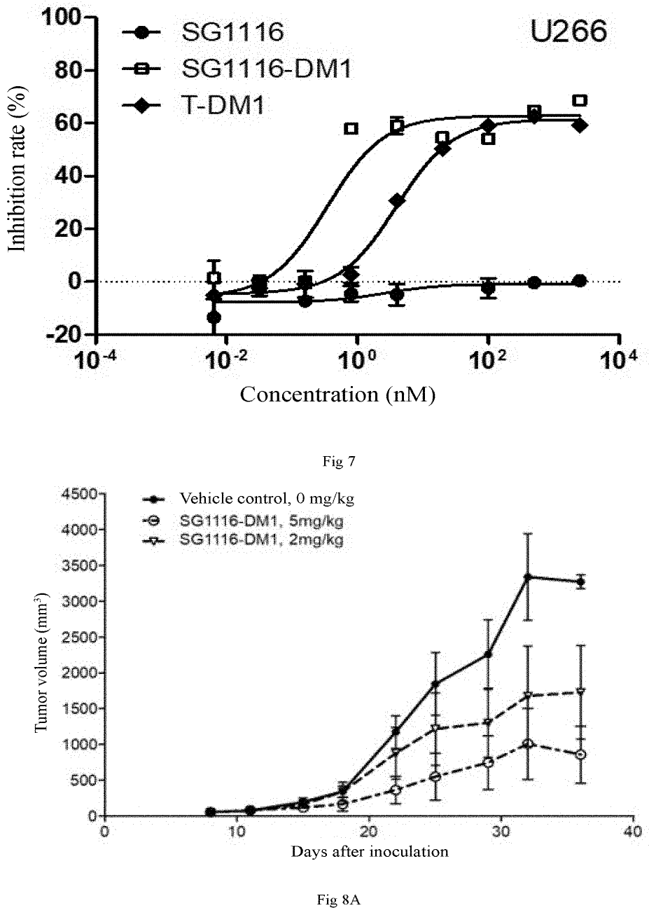

The results are shown in FIG. 7. The results show that, the SG1116 antibody conjugated with maytansine derivative DM1 (SG1116DM1) shows significant inhibition effect on the proliferation of BCMA-positive myeloma cells U266, of which the IC₅₀ is 0.3473 nM, and the IC₅₀ of T-DM1 (Kadcyla™) is 3.951 nM.

### Example 11 Activity of Antibody-Conjugated Maytansine Derivative DM1 to Inhibit Tumor In Vivo

With NCI-H929 cells as representative cells, a tumor-bearing animal model was established to evaluate the activity of SG1116-DM1 to inhibit the tumor in vivo.

Female, 6-7-week-old NOD/SCID mice (Shanghai Lingchang Biotech Co., Ltd.) were selected, and NCI-H929 cells were cultured in RPM11640+0.05 mM 3-ME culture solution containing 10% of fetal calf serum. NCI-H929 cells at the exponential growth phase were collected, resuspended in PBS to a suitable concentration and mixed with matrigel at 1:1 for subcutaneous tumor inoculation on the right back of mice. After inoculation, when the average tumor volume was 54 mm³, mice were randomly divided into 3 groups according to the tumor size (a vehicle control group, a SG1116-DM1 high-dose group, and a SG1116-DM1 low-dose group), with 6 mice in each group. These mice were administered by intraperitoneal injection, at a dose of 5 mg/kg per dose for the high-dose group and at a dose of 2 mg/kg per dose for the low-dose group, and administered once every two weeks for totally two times. The experiment ended two weeks after the last administration. When the tumor volume of a single mouse exceeded 3000 mm³, the mouse was euthanized. The tumor growth of mice was observed during administration. The efficacy was evaluated according to the relative tumor growth inhibition rate (TGI), and the safety was evaluated according to the change of animal weight and the mortality. The relative tumor growth inhibition rate TGI (%) was calculated following the equation as below: TGI %=(1−T/C)×100%. (T and C are the relative tumor volume (RTV) or tumor weight (TW) of the treatment group and the control group at a certain time point, respectively).

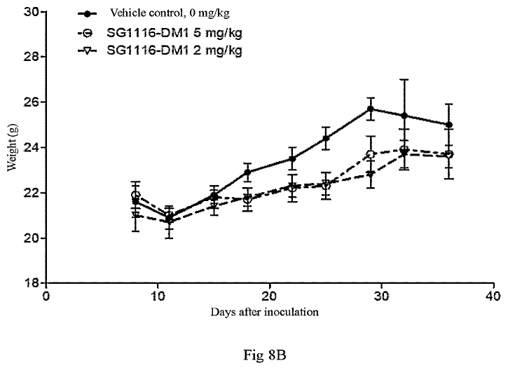

The results are shown in FIGS. 8A-8B. Both the high-dose group and the low-dose group of SG1116-DM1 (5 mg/kg, 2 mg/kg) showed obvious anti-tumor effects on the 17th day after grouping, for which the relative tumor growth inhibition rates TGI (%) were 70.13% and 34.06% respectively, where there is a statistically significant difference for the high-dose group (5 mg/kg) relative to the vehicle control group (p value <0.05), and there is no statistically significant difference for the low-dose group (2 mg/kg) relative to the vehicle control group.

On the 28th day after grouping (4 mice in the vehicle control group, 1 mouse in the SG1116-DM1 high-dose group, and 2 mice in the SG1116-DM1 low-dose group respectively were euthanized because their tumor volume exceeded 3000 mm³, and the surviving mice were analyzed statistically): the average tumor volume for the SG1116-DM1 high-dose group (5 mg/kg) was 857.74 mm³, the relative tumor growth inhibition rate TGI (%) was 73.78%, with a statistically significant difference relative to the control group (p value <0.01). The average tumor volume for the SG1116-DM1 low-dose group (2 mg/kg) was 1727.89 mm³, the relative tumor growth inhibition rate TGI (%) was 47.18%, with no statistically significant difference relative to the control group.

The SG1116-DM1 treatment groups had significant anti-tumor effects and are dose-dependent.

No animals died during the treatment, no obvious drug toxicity was shown and the tolerance was good.

The foregoing detailed description is provided by way of explanation and examples, and is not intended to limit the scope of the appended claims. Various changes of the embodiments currently listed herein are obvious to those of ordinary skills in the art, and are reserved within the scope of the appended claims and their equivalents.

1. An antibody or an antigen-binding fragment thereof, comprising:
at least one CDR in an antibody light chain variable region VL, wherein the VL comprises an amino acid sequence as set forth in SEQ ID NO: 7, and
at least one CDR in an antibody heavy chain variable region VH, wherein the VH comprises an amino acid sequence as set forth in SEQ ID NO: 8.

2-4. (canceled)

5. The antibody or the antigen-binding fragment thereof according to claim 1, wherein the antibody is selected from the group consisting of a monoclonal antibody, a single-chain antibody, a chimeric antibody, a multispecific antibody, a humanized antibody, and a fully human antibody, and
wherein the antigen-binding fragment is selected from the group consisting of Fab, Fab′, F(ab)2, F(ab′)2, Fv, and ScFv.

6. (canceled)

7. The antibody or the antigen-binding fragment thereof according to claim 1, competing with a reference antibody for binding to the BCMA protein, wherein:
the reference antibody comprises a light chain variable region and a heavy chain variable region,
the light chain variable region of the reference antibody comprises LCDR1, LCDR2, and LCDR3,
the LCDR1 comprises an amino acid sequence as set forth in SEQ ID NO: 1;
the LCDR2 comprises an amino acid sequence as set forth in SEQ ID NO: 2;
the LCDR3 comprises an amino acid sequence as set forth in SEQ ID NO: 3;
the heavy chain variable region of the reference antibody comprises HCDR1, HCDR2, and HCDR3,
the HCDR1 comprises an amino acid sequence as set forth in SEQ ID NO: 4;
the HCDR2 comprises an amino acid sequence as set forth in SEQ ID NO: 5; and
the HCDR3 comprises an amino acid sequence as set forth in SEQ ID NO: 6.

8-10. (canceled)

11. The antibody or the antigen-binding fragment thereof according to claim 1, wherein:
the antibody comprises an antibody light chain or a fragment thereof;
the antibody light chain or the fragment thereof comprises LCDR1, LCDR2, and LCDR3,
the LCDR1 comprises an amino acid sequence as set forth in SEQ ID NO: 1;
the LCDR2 comprises an amino acid sequence as set forth in SEQ ID NO: 2; and
the LCDR3 comprises an amino acid sequence as set forth in SEQ ID NO: 3.

12-14. (canceled)

15. The antibody or the antigen-binding fragment thereof according to claim 11, wherein the antibody light chain or the fragment thereof comprises a light chain variable region VL, and
wherein the light chain variable region VL comprises an amino acid sequence as set forth in SEQ ID NO: 7.

16-17. (canceled)

18. The antibody or the antigen-binding fragment thereof according to claim 11, wherein the antibody light chain or the fragment thereof comprises an amino acid sequence as set forth in SEQ ID NO: 9.

19. The antibody or the antigen-binding fragment thereof according to claim 1, wherein:
the antibody comprises an antibody heavy chain or a fragment thereof;
the antibody heavy chain or the fragment thereof comprises HCDR1, HCDR2, and HCDR3;
the HCDR2 comprises an amino acid sequence as set forth in SEQ LID NO: 4;
the HCDR2 comprises an amino acid sequence as set forth in SEQ ID NO: 5; and
the HCDR3 comprises an amino acid sequence as set forth in SEQ ID NO: 6.

20-22. (canceled)

23. The antibody or the antigen-binding fragment thereof according to claim 19, wherein the antibody heavy chain or the fragment thereof comprises a heavy chain variable region VH, and
wherein the heavy chain variable region VH comprises an amino acid sequence as set forth in SEQ ID NO: 8.

24-26. (canceled)

27. The antibody or the antigen-binding fragment thereof according to claim 19, wherein the antibody heavy chain comprises an amino acid sequence as set forth in SEQ ID NO: 10.

28. (canceled)

29. One or more isolated nucleic acid molecules, encoding the antibody or the antigen-binding fragment thereof according to claim 1.

30. One or more vectors, comprising the one or more nucleic acid molecules according to claim 29.

31. A cell, comprising the one or more nucleic acid molecules according to claim 29.

32. (canceled)

33. An immunoconjugate, comprising the antibody or the antigen-binding fragment thereof according to claim 1.

34. The immunoconjugate according to claim 33, further comprising at least one additional reagent selected from the group consisting of a chemotherapeutic agent, a radioactive element, a cell growth inhibitor, and a cytotoxic agent.

35-36. (canceled)

37. The immunoconjugate according to claim 34, wherein the at least one additional reagent comprises maytansine or a derivative thereof.

38. (canceled)

39. A pharmaceutical composition, comprising:
the antibody or the antigen-binding fragment thereof according to claim 1, and
optionally a pharmaceutically acceptable adjuvant.

40-47. (canceled)

48. A method of preventing or treating a tumor in a subject in need thereof, comprising administering to the subject the antibody or the antigen-binding fragment thereof according to claim 1.

49. The method according to claim 48, wherein the tumor comprises a BCMA positive tumor.

50. (canceled)

51. The method according to claim 48, wherein the tumor comprises a tumor selected from the group consisting of myeloma, lymphoma, and hematologic malignancy.

52-53. (canceled)

54. A method of judging diseases or conditions related to the expression of a BCMA protein in a subject, comprising:
contacting a sample derived from the subject with the antibody or the antigen-binding fragment thereof according to claim 1, and
determining the presence and/or content of substances in the sample which are capable of specifically binding to the antibody or the antigen-binding fragment thereof.


SEQUENCE LISTING (12 sequences)

SEQ ID NO: 1 - protein, 15 aa, Artificial Sequence
  Synthetic Peptide, SG1116 LCDR1
RASESVTILGSHLIY

SEQ ID NO: 2 - protein, 7 aa, Artificial Sequence
  Synthetic Peptide, SG1116 LCDR2
LASNVQT

SEQ ID NO: 3 - protein, 9 aa, Artificial Sequence
  Synthetic Peptide, SG1116 LCDR3
LQSRTIPRT

SEQ ID NO: 4 - protein, 5 aa, Artificial Sequence
  Synthetic Peptide, SG1116 HCDR1
RYSMN

SEQ ID NO: 5 - protein, 17 aa, Artificial Sequence
  Synthetic Peptide, SG1116 HCDR2
RINTGSGAPIYADDFKG

SEQ ID NO: 6 - protein, 8 aa, Artificial Sequence
  Synthetic Peptide, SG1116 HCDR3
DYNYSLDH

SEQ ID NO: 7 - protein, 111 aa, Artificial Sequence
  Synthetic Peptide, SG1116 light chain variable region
DIVLTQSPPSLAMSLGKRATISCRASESVTILGSHLIYWYQQKPGQPPTLLIQLASNVQTGVPARFSGSGSRTDFTLTIDPVEEDDVAVYYCLQSRTIPRTFGGGTKLEIK

SEQ ID NO: 8 - protein, 117 aa, Artificial Sequence
  Synthetic Peptide, SG1116 heavy chain variable region
QVQLVQSGPELKKPGETVKISCKASGYTFSRYSMNWVRQAPGKGLEWMGRINTGSGAPIYADDFKGRFTFSVDTSASTAYLVINNLKSEDTASYFCSNDYNYSLDHWGQGTAVTVSS

SEQ ID NO: 9 - protein, 218 aa, Artificial Sequence
  Synthetic Peptide, SG1116 light chain
DIVLTQSPPSLAMSLGKRATISCRASESVTILGSHLIYWYQQKPGQPPTLLIQLASNVQTGVPARFSGSGSRTDFTLTIDPVEEDDVAVYYCLQSRTIPRTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 10 - protein, 447 aa, Artificial Sequence
  Synthetic Peptide, SG1116 heavy chain
QVQLVQSGPELKKPGETVKISCKASGYTFSRYSMNWVRQAPGKGLEWMGRINTGSGAPIYADDFKGRFTFSVDTSASTAYLVINNLKSEDTASYFCSNDYNYSLDHWGQGTAVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 107 aa, Artificial Sequence [sequence omitted]

SEQ ID NO: 12 - protein, 330 aa, Artificial Sequence [sequence omitted]